(19) United States
(12) Patent Application Publication
(10) Pub. No.: US 2022/0031182 A1
(43) Pub. Date: Feb. 3, 2022

(54) DETECTION DEVICE

(71) Applicants: Japan Display Inc., Tokyo (JP); The University of Tokyo, Tokyo (JP)

(72) Inventors: Hirofumi KATO, Tokyo (JP); Takanori TSUNASHIMA, Tokyo (JP); Makoto UCHIDA, Tokyo (JP); Takashi NAKAMURA, Tokyo (JP); Akio TAKIMOTO, Tokyo (JP); Takao SOMEYA, Tokyo (JP); Tomoyuki YOKOTA, Tokyo (JP)

(21) Appl. No.: 17/500,179

(22) Filed: Oct. 13, 2021

Related U.S. Application Data
(63) Continuation of application No. PCT/JP2020/016503, filed on Apr. 15, 2020.

(30) Foreign Application Priority Data
Apr. 17, 2019 (JP) 2019-078925

(51) Int. Cl. A61B 5/024; A61B 5/0285; A61B 5/00

(52) U.S. Cl. CPC A61B 5/02433; G06K 9/0004; A61B 5/7278; A61B 5/0285

(57) ABSTRACT

A detection device includes a first optical sensor, a second optical sensor disposed at a predetermined distance from the first optical sensor, a light source that emits light to be detected by the first optical sensor and the second optical sensor facing a living body tissue including a blood vessel, and a processor that calculates a pulse wave velocity of the blood vessel based on a time-series variation of an output of the first optical sensor, a time-series variation of an output of the second optical sensor, and the predetermined distance.

### CROSS-REFERENCE TO RELATED APPLICATION

This application claims the benefit of priority from Japanese Patent Application No. 2019-078925 filed on Apr. 17, 2019 and International Patent Application No. PCT/JP2020/016503 filed on Apr. 15, 2020, the entire contents of which are incorporated herein by reference.

### BACKGROUND

### 1. Technical Field

What is disclosed herein relates to a detection device.

### 2. Description of the Related Art

Optical sensors capable of detecting a fingerprint pattern and/or a vascular pattern are known (for example, Japanese Patent Application Laid-open Publication No. 2009-032005).

It is desired to obtain a pulse wave velocity using such an optical sensor.

For the foregoing reasons, there is a need for a detection device capable of obtaining the pulse wave velocity.

### SUMMARY

According to an aspect, a detection device includes: a first optical sensor; a second optical sensor disposed at a predetermined distance from the first optical sensor; a light source configured to emit light to be detected by the first optical sensor and the second optical sensor facing a living body tissue including a blood vessel; and a processor configured to calculate a pulse wave velocity of the blood vessel based on a time-series variation of an output of the first optical sensor, a time-series variation of an output of the second optical sensor, and the predetermined distance.

### BRIEF DESCRIPTION OF THE DRAWINGS

FIG. 1 is a plan view illustrating a detection device according to an embodiment;

FIG. 2 is a block diagram illustrating a configuration example of the detection device according to the embodiment;

FIG. 3 is a circuit diagram illustrating the detection device;

FIG. 4 is a circuit diagram illustrating a plurality of partial detection areas;

FIG. 5 is a sectional view illustrating a schematic sectional configuration of a sensor;

FIG. 6 is a graph schematically illustrating a relation between a wavelength and a conversion efficiency of light incident on a photodiode;

FIG. 7 is a timing waveform diagram illustrating an operation example of the detection device;

FIG. 8 is a timing waveform diagram illustrating an operation example during a reading period in FIG. 7;

FIG. 9 is an explanatory diagram for explaining a relation between driving of the sensor and lighting operations of light sources in the detection device;

FIG. 10 is an explanatory diagram for explaining a relation between the driving of the sensor and the lighting operations of the light sources according to a first modification of the embodiment;

FIG. 11 is a schematic view illustrating an exemplary positional relation between second light sources, the sensor, and a blood vessel in a finger;

FIG. 12 is a schematic view illustrating a plurality of points in a photodiode that are exemplarily set when a planar detection area formed by a plurality of photodiodes provided so as to face the finger is viewed in a plan view;

FIG. 13 is a flowchart illustrating an exemplary flow of processing for correcting a temporal shift that branches in accordance with a control mode of a lighting time of the light sources;

FIG. 14 is a timing diagram for explaining temporal shifts of effective exposure periods and output timings when a reset period and the reading period overlap a lighting period of the second light sources;

FIG. 15 is a timing diagram for explaining the temporal shifts of the output timings when the reset period and the reading period do not overlap the lighting period of the second light sources;

FIG. 16 is an explanatory diagram illustrating examples of temporal shifts of outputs from the respective photodiodes before and after correction;

FIG. 17 is a schematic view illustrating a main configuration example of a detection device in a form wearable on a wrist;

FIG. 18 is a schematic diagram illustrating an example of detection of a pulse wave velocity of the blood vessel by the detection device illustrated in FIG. 17;

FIG. 19 is a diagram illustrating an arrangement example of the sensor of the detection device mounted on a bandanna;

FIG. 20 is a diagram illustrating an arrangement example of the sensor of the detection device mounted on clothes; and

FIG. 21 is a diagram illustrating an arrangement example of the sensor of the detection device mounted on an adhesive sheet.

### DETAILED DESCRIPTION

The following describes a mode (an embodiment) for carrying out the present invention in detail with reference to the drawings. The present invention is not limited to the description of the embodiment given below. Components described below include those easily conceivable by those skilled in the art or those substantially identical thereto. Moreover, the components described below can be appropriately combined. The disclosure is merely an example, and the present invention naturally encompasses appropriate modifications easily conceivable by those skilled in the art while maintaining the gist of the invention. To further clarify the description, the drawings schematically illustrate, for example, widths, thicknesses, and shapes of various parts as compared with actual aspects thereof, in some cases. However, they are merely examples, and interpretation of the present invention is not limited thereto. The same element as that illustrated in a drawing that has already been discussed is denoted by the same reference numeral through the description and the drawings, and detailed description thereof will not be repeated in some cases where appropriate.

In this disclosure, when an element is described as being “on” another element, the element can be directly on the other element, or there can be one or more elements between the element and the other element.

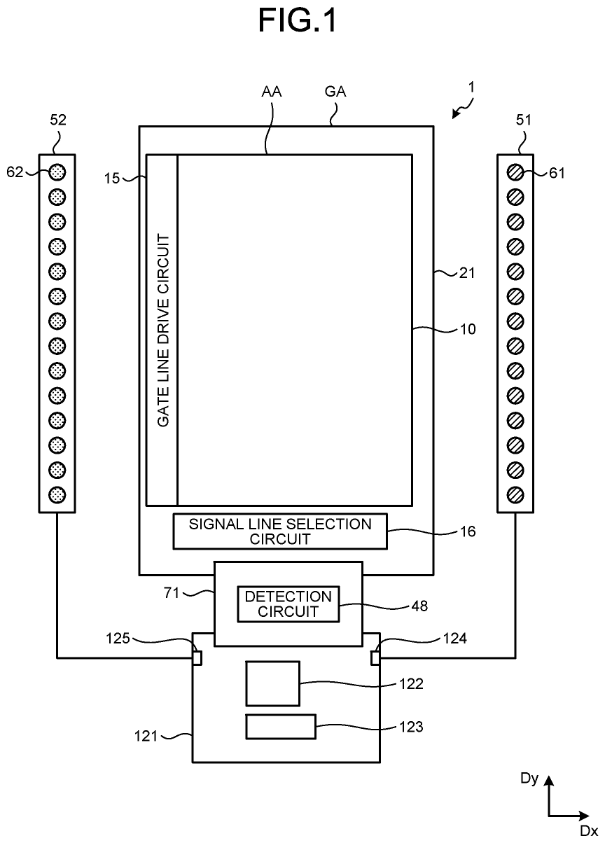

FIG. 1 is a plan view illustrating a detection device according to the embodiment. As illustrated in FIG. 1, a detection device 1 includes a sensor base member 21, a sensor 10, a gate line drive circuit 15, a signal line selection circuit 16, a detection circuit 48, a control circuit 122, a power supply circuit 123, a first light source base member 51, a second light source base member 52, at least one first light source 61, and at least one second light source 62. While the embodiment exemplifies a plurality of types of light sources (the first light sources 61 and the second light sources 62) as light sources, the light sources may be of one type.

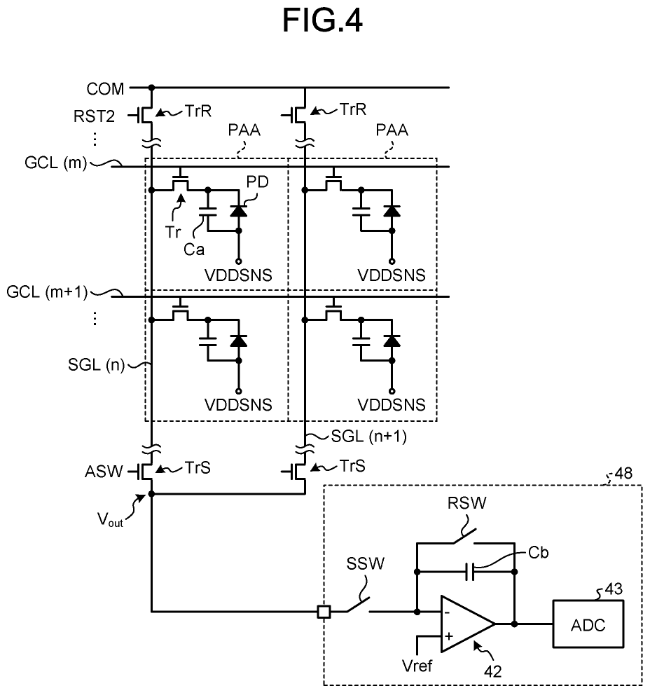

A control board 121 is electrically coupled to the sensor base member 21 through a flexible printed circuit board 71. The flexible printed circuit board 71 is provided with the detection circuit 48. The control board 121 is provided with the control circuit 122 and the power supply circuit 123. The control circuit 122 is, for example, a field programmable gate array (FPGA). The control circuit 122 supplies control signals to the sensor 10, the gate line drive circuit 15, and the signal line selection circuit 16 to control a detection operation of the sensor 10. The control circuit 122 supplies control signals to the first light sources 61 and the second light sources 62 to control to turn on and off the first light sources 61 and the second light sources 62. The power supply circuit 123 supplies voltage signals including, for example, a sensor power supply signal VDDSNS (refer to FIG. 4) to the sensor 10, the gate line drive circuit 15, and the signal line selection circuit 16. The power supply circuit 123 also supplies a power supply voltage to the first light sources 61 and the second light sources 62.

The sensor base member 21 has a detection area AA and a peripheral area GA. The detection area AA is an area provided with a plurality of photodiodes PD (refer to FIG. 4) included in the sensor 10. The peripheral area GA is an area between the outer circumference of the detection area AA and ends of the sensor base member 21 and is an area not overlapping the photodiodes PD.

The gate line drive circuit 15 and the signal line selection circuit 16 are provided in the peripheral area GA. Specifically, the gate line drive circuit 15 is provided in an area of the peripheral area GA extending along a second direction Dy, and the signal line selection circuit 16 is provided in an area of the peripheral area GA extending along a first direction Dx and is provided between the sensor 10 and the detection circuit 48.

The first direction Dx is a direction in a plane parallel to the sensor base member 21. The second direction Dy is a direction in a plane parallel to the sensor base member 21 and is a direction orthogonal to the first direction Dx. The second direction Dy may intersect the first direction Dx without being orthogonal thereto. A third direction Dz is a direction orthogonal to the first direction Dx and the second direction Dy, and is the normal direction of the sensor base member 21.

The first light sources 61 are provided on the first light source base member 51 and are arranged along the second direction Dy. The second light sources 62 are provided on the second light source base member 52, and are arranged along the second direction Dy. The first light source base member 51 and the second light source base member 52 are electrically coupled through terminals 124 and 125, respectively, provided on the control board 121 to the control circuit 122 and the power supply circuit 123.

For example, inorganic light-emitting diodes (LEDs) or organic electroluminescent (EL) diodes (organic light-emitting diodes) (OLEDs) are used as the first light sources 61 and the second light sources 62. The first light sources 61 and the second light sources 62 emit first light L61 (refer to FIG. 18) and second light L62 (refer to, for example, FIG. 11), respectively, having wavelengths different from each other. The first light L61 and the second light L62 have different maximum emission wavelengths from each other. The term “maximum emission wavelength” refers to a wavelength that exhibits the maximum emission intensity in an emission spectrum representing a relation between the wavelength and the emission intensity of each of the first light L61 and the second light L62. Hereinafter, when a value of the wavelength is simply mentioned, the mentioned value refers to an assumed maximum emission wavelength.

The first light L61 emitted from the first light sources 61 is mainly reflected on a surface of a detection target object, for example, a finger Fg, and enters the sensor 10. Thus, the sensor 10 can detect a fingerprint by detecting a shape of asperities of the surface of, for example, the finger Fg. The second light L62 emitted from the second light sources 62 is mainly reflected inside, for example, the finger Fg, or transmitted through, for example, the finger Fg, and enters the sensor 10. Thus, the sensor 10 can detect biological information on the inside, for example, the finger Fg. The biological information is, for example, a pulse wave, pulsation, and a blood vessel image of the finger Fg or a palm.

As an example, the first light L61 may have a wavelength in a range from 520 nm to 600 nm, for example, at approximately 560 nm, and the second light L62 may have a wavelength in a range from 780 nm to 900 nm, for example, at approximately 850 nm. In this case, the first light L61 is blue or green visible light, and the second light L62 is infrared light. The sensor 10 can detect a fingerprint based on the first light L61 emitted from the first light sources 61. The second light L62 emitted from the second light sources 62 is reflected in the detection target object such as the finger Fg, or transmitted through or absorbed by, for example, the finger Fg, and enters the sensor 10. Thus, the sensor 10 can detect the pulse wave and the blood vessel image (vascular pattern) as the biological information on the inside, for example, the finger Fg.

Alternatively, the first light L61 may have a wavelength in a range from 600 nm to 700 nm, for example, at approximately 660 nm, and the second light L62 may have a wavelength in a range from 780 nm to 900 nm, for example, at approximately 850 nm. In this case, the sensor 10 can detect a blood oxygen saturation level in addition to the pulse wave, the pulsation, and the blood vessel image as the biological information based on the first light L61 emitted from the first light sources 61 and the second light L62 emitted from the second light sources 62. In this manner, since the detection device 1 includes the first light sources 61 and the second light sources 62, the detection device 1 can detect the various types of the biological information by performing the detection based on the first light L61 and the detection based on the second light L62.

The arrangement of the first light sources 61 and the second light sources 62 illustrated in FIG. 1 is merely an example, and can be changed as appropriate. For example, the first light sources 61 and the second light sources 62 may be arranged on each of the first light source base member 51 and the second light source base member 52. In this case, a group including the first light sources 61 and a group including the second light sources 62 may be arranged in the second direction Dy, or the first light source 61 and the second light source 62 may be alternately arranged in the second direction Dy. The number of the light source base members provided with the first light sources 61 and the second light sources 62 may be one, or three or more.

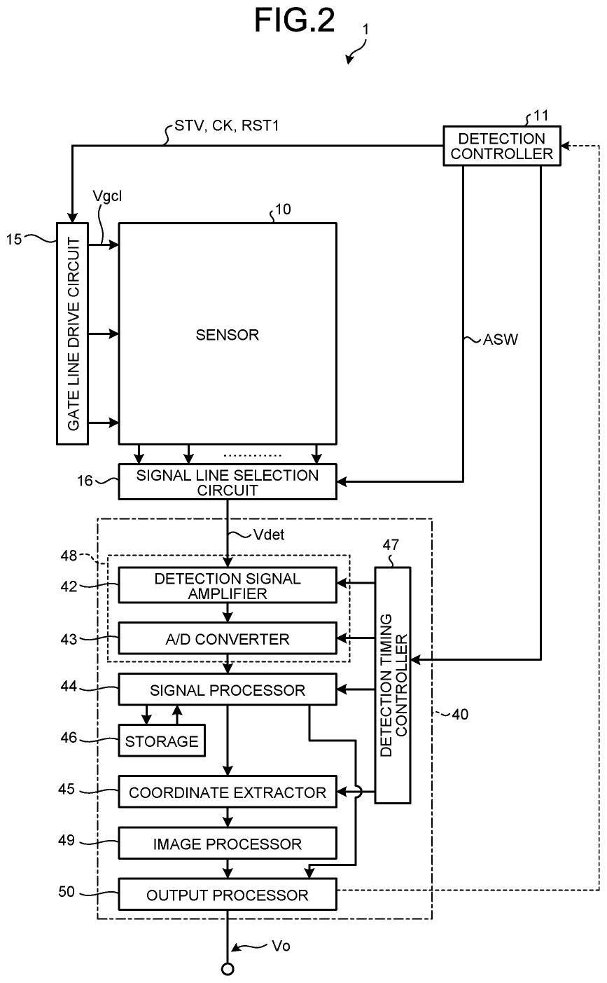

FIG. 2 is a block diagram illustrating a configuration example of the detection device according to the embodiment. As illustrated in FIG. 2, the detection device 1 further includes a detection controller 11 and a detector 40. The control circuit 122 includes some or all functions of the detection controller 11. The control circuit 122 also includes some or all functions of the detector 40 except those of the detection circuit 48.

The sensor 10 is an optical sensor including the photodiodes PD serving as photoelectric conversion elements. Each of the photodiodes PD included in the sensor 10 outputs an electrical signal corresponding to light emitted thereto to the signal line selection circuit 16. The signal line selection circuit 16 sequentially selects a signal line SGL in response to a selection signal ASW from the detection controller 11. As a result, the electrical signal is output as a detection signal Vdet to the detector 40. The sensor 10 performs the detection in response to a gate drive signal Vgcl supplied from the gate line drive circuit 15.

The detection controller 11 is a circuit that supplies respective control signals to the gate line drive circuit 15, the signal line selection circuit 16, and the detector 40 to control operations thereof. The detection controller 11 supplies various control signals including, for example, a start signal STV, a clock signal CK, and a reset signal RST1 to the gate line drive circuit 15. The detection controller 11 also supplies various control signals including, for example, the selection signal ASW to the signal line selection circuit 16. The detection controller 11 also supplies various control signals to the first light sources 61 and the second light sources 62 to control to turn on and off the first light sources 61 and the second light sources 62.

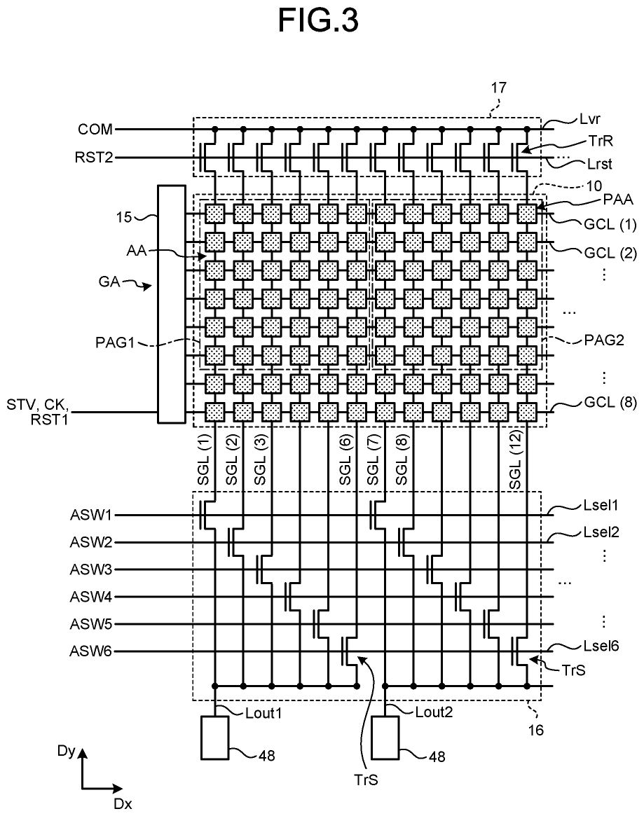

The gate line drive circuit 15 is a circuit that drives a plurality of gate lines GCL (refer to FIG. 3) based on the various control signals. The gate line drive circuit 15 sequentially or simultaneously selects the gate lines GCL and supplies the gate drive signals Vgcl to the selected gate lines GCL. Through this operation, the gate line drive circuit 15 selects the photodiodes PD coupled to the gate lines GCL.

The signal line selection circuit 16 is a switch circuit that sequentially or simultaneously selects a plurality of signal lines SGL (refer to FIG. 3). The signal line selection circuit 16 is, for example, a multiplexer. The signal line selection circuit 16 couples the selected signal lines SGL to the detection circuit 48 based on the selection signal ASW supplied from the detection controller 11. Through this operation, the signal line selection circuit 16 outputs the detection signal Vdet of each of the photodiodes PD to the detector 40.

The detector 40 includes the detection circuit 48, a signal processor 44, a coordinate extractor 45, a storage 46, a detection timing controller 47, an image processor 49, and an output processor 50. Based on a control signal supplied from the detection controller 11, the detection timing controller 47 controls the detection circuit 48, the signal processor 44, the coordinate extractor 45, and the image processor 49 so as to operate in synchronization with one another.

The detection circuit 48 is, for example, an analog front end (AFE) circuit. The detection circuit 48 is, for example, a signal processing circuit having functions of a detection signal amplifier 42 and an analog-to digital (A/D) converter 43. The detection signal amplifier 42 amplifies the detection signal Vdet. The A/D converter 43 converts an analog signal output from the detection signal amplifier 42 into a digital signal.

The signal processor 44 is a logic circuit that detects a predetermined physical quantity received by the sensor 10 based on an output signal of the detection circuit 48. When the finger Fg is in contact with or in proximity to the detection area AA, the signal processor 44 can detect the asperities on the surface of the finger Fg or the palm based on the signal from the detection circuit 48. The signal processor 44 can also detect the biological information based on the signal from the detection circuit 48. The biological information is, for example, the blood vessel image, a pulse wave, the pulsation, and/or the blood oxygen saturation level of the finger Fg or the palm.

In the case of obtaining the human blood oxygen saturation level, for example, 660 nm (the range is from 500 nm to 700 nm) is employed as the first light L61, and approximately 850 nm (the range is from 800 nm to 930 nm) is employed as the second light L62. Since the amount of light absorption changes with an amount of oxygen taken up by hemoglobin, the photodiode PD detects an amount of light obtained by subtracting the amount of light absorbed by the blood (hemoglobin) from that of each of the first light L61 and the second light L62 that have been emitted. Most of the oxygen in the blood is reversibly bound to hemoglobin in red blood cells, and a small portion of the oxygen is dissolved in blood plasma. More specifically, the value of percentage of oxygen with respect to an allowable amount thereof in the blood as a whole is called the oxygen saturation level (SpO₂). The blood oxygen saturation level can be calculated from the amount of light obtained by subtracting the amount of light absorbed by the blood (hemoglobin) from that of the light emitted at the two wavelengths of the first light L61 and the second light L62.

The signal processor 44 may acquire the detection signals Vdet (biological information) simultaneously detected by the photodiodes PD, and average the detection signals Vdet. In this case, the detector 40 can perform the stable detection by reducing a measurement error caused by noise or a relative displacement between the detection target object such as the finger Fg and the sensor 10.

The storage 46 temporarily stores therein a signal calculated by the signal processor 44. The storage 46 may be, for example, a random access memory (RAM) or a register circuit.

The coordinate extractor 45 is a logic circuit that obtains, when the contact or the proximity of the finger is detected by the signal processor 44, detection coordinates of the asperities on the surface of, for example, the finger. The coordinate extractor 45 is also a logic circuit that obtains detected coordinates of blood vessels of the finger Fg or the palm. The image processor 49 combines the detection signals Vdet output from the respective photodiodes PD of the sensor 10 to generate two-dimensional information representing the shape of the asperities on the surface of, for example, the finger Fg and two-dimensional information representing a shape of the blood vessels of the finger Fg or the palm. The coordinate extractor 45 and the image processor 49 may be omitted.

The output processor 50 serves as a processor for performing processing based on the output from the photodiodes PD. Specifically, the output processor 50 of the embodiment outputs at least a sensor output Vo including at least pulse wave data based on the detection signal Vdet acquired through the signal processor 44. In the embodiment, the signal processor 44 outputs data indicating a variation (amplitude) in output of the detection signal Vdet of each of the photodiodes PD (to be described later), and the output processor 50 determines which output is to be employed as the sensor output Vo. However, the signal processor 44 or the output processor 50 may perform both the above-described operations. The output processor 50 may include, for example, the detected coordinates obtained by the coordinate extractor 45 and the two-dimensional information generated by the image processor 49 in the sensor output Vo. The function of the output processor 50 may be integrated in another component (for example, the image processor 49).

When the detection device of, for example, the pulse wave is mounted on a human body, noise is also detected associated with, for example, breathing, a change in attitude of the human body, and/or motion of the human body. Therefore, the signal processor 44 may be provided with a noise filter as required. The noise generated by the breathing and/or the change in attitude has frequency components of, for example, 1 Hz or lower, which are sufficiently lower than frequency components of the pulse wave. Therefore, the noise can be removed by using a band-pass filter as the noise filter. The band-pass filter may be provided, for example, in a detection signal amplifier 42. The frequency components of the noise generated by the motion of the human body are, for example, from several hertz to 100 hertz, and may overlap the frequency components of the pulse wave. In this case, however, the frequency is not constant and has a frequency fluctuation. Therefore, a noise filter is used that removes noise the frequencies of which have fluctuation components. As an example of a method for removing the frequencies having fluctuation components (first method for removing fluctuation components), a property may be used that a time lag of a peak value of the pulse wave occurs depending on the place of measurement of the human body. That is, the pulse wave has a time lag depending on the place of measurement of the human body, while the noise generated by the motion of the human body or the like has no time lag or a time lag smaller than that of the pulse wave. Therefore, the pulse wave is measured in at least two different places, and if peak values measured in the different places have occurred within a predetermined time, the pulse wave is removed as noise. Even in this case, a case can be considered where the waveform caused by noise accidentally overlaps the waveform caused by the pulse wave. However, in this case, the two waveforms overlap each other at only one place of the different places. Therefore, the waveform caused by noise can be distinguished from the waveform caused by the pulse wave. For example, the signal processor 44 can perform this processing. As another example of the method for removing the frequencies having fluctuation components (second method for removing fluctuation components), the signal processor 44 removes frequency components having different phases. In this case, for example, a short-time Fourier transform may be performed to remove the fluctuation components, and then, an inverse Fourier transform may be performed. Moreover, a commercial frequency power supply (50 Hz or 60 Hz) also serves as a noise source. However, in this case as well, in the same manner as the noise generated by the motion of the human body or other factors, the peak values measured at the different places have no time lag therebetween or a time lag therebetween smaller than that of the pulse wave. Therefore, the noise can be removed using the same method as the above-described first method for removing fluctuation components. Alternatively, the noise generated by the commercial frequency power supply may be removed by providing a shield on a surface on the opposite side of a detection surface of a detecting element.

The following describes a circuit configuration example of the detection device 1. FIG. 3 is a circuit diagram illustrating the detection device. FIG. 4 is a circuit diagram illustrating a plurality of partial detection areas. FIG. 4 also illustrates a circuit configuration of the detection circuit 48.

As illustrated in FIG. 3, the sensor 10 has a plurality of partial detection areas PAA arranged in a matrix having a row-column configuration. Each of the partial detection areas PAA is provided with the photodiode PD.

The gate lines GCL extend in the first direction Dx, and are coupled to the partial detection areas PAA arranged in the first direction Dx. A plurality of gate lines GCL(1), GCL(2), . . . , GCL(8) are arranged in the second direction Dy, and are each coupled to the gate line drive circuit 15. In the following description, the gate lines GCL(1), GCL(2), . . . , GCL(8) will each be simply referred to as the gate line GCL when they need not be distinguished from one another. For ease of understanding of the description, FIG. 3 illustrates eight gate lines GCL. However, this is merely an example, and M gate lines GCL (where M is eight or larger, and is, for example, 256) may be arranged.

The signal lines SGL extend in the second direction Dy and are coupled to the photodiodes PD of the partial detection areas PAA arranged in the second direction Dy. A plurality of signal lines SGL(1), SGL(2), . . . , SGL(12) are arranged in the first direction Dx, and are each coupled to the signal line selection circuit 16 and a reset circuit 17. In the following description, the signal lines SGL(1), SGL(2), . . . , SGL(12) will each be simply referred to as the signal line SGL when need not be distinguished from one another.

For ease of understanding of the description, 12 of the signal lines SGL are illustrated. However, this is merely an example, and N signal lines SGL (where N is 12 or larger, and is, for example, 252) may be arranged. The resolution of the sensor is, for example, 508 dots per inch (dpi), and the number of cells is 252×256. In FIG. 3, the sensor 10 is provided between the signal line selection circuit 16 and the reset circuit 17. The configuration is not limited thereto. The signal line selection circuit 16 and the reset circuit 17 may be coupled to ends of the signal lines SGL in the same direction. One sensor has an area of substantially 50×50 μm², for example, and the detection area AA has an area of, for example, 12.6×12.8 mm².

The gate line drive circuit 15 receives the various control signals such as the start signal STV, the clock signal CK, and the reset signal RST1 from the control circuit 122 (refer to FIG. 1). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and corresponding ones of the partial detection areas PAA arranged in the first direction Dx are selected as detection targets.

The gate line drive circuit 15 may perform different driving for each of detection modes including the detection of a fingerprint and the detection of different items of the biological information (such as the pulse wave, the pulsation, the blood vessel image, and the blood oxygen saturation level). For example, the gate line drive circuit 15 may drive more than one gate line GCL collectively.

Specifically, the gate line drive circuit 15 may simultaneously select a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6) and supplies thereto the gate drive signals Vgcl. The gate line drive circuit 15 supplies the gate drive signals Vgcl through the selected six gate lines GCL to the first switching elements Tr. Through this operation, group areas PAG1 and PAG2 each including more than one partial detection area PAA arranged in the first direction Dx and the second direction Dy are selected as the respective detection targets. The gate line drive circuit 15 drives the predetermined number of the gate lines GCL collectively, and sequentially supplies the gate drive signals Vgcl to the gate lines GCL in units of the predetermined number of the gate lines GCL. Hereinafter, when positions of different group areas such as the group areas PAG1 and PAG2 are not distinguished from each other, each of the group areas will be called “group area PAG”.

The signal line selection circuit 16 includes a plurality of selection signal lines Lsel, a plurality of output signal lines Lout, and third switching elements TrS. The third switching elements TrS are provided correspondingly to the signal lines SGL. Six signal lines SGL(1), SGL(2), . . . , SGL(6) are coupled to a common output signal line Lout1. Six signal lines SGL(7), SGL(8), . . . , SGL(12) are coupled to a common output signal line Lout2. The output signal lines Lout1 and Lout2 are each coupled to the detection circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the third switching elements TrS included in one of the signal line blocks, respectively. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . . . , SGL(6), respectively. The selection signal line Lsel1 is coupled to the third switching element TrS corresponding to the signal line SGL(1) and the third switching element TrS corresponding to the signal line SGL(7). The selection signal line Lsel2 is coupled to the third switching element TrS corresponding to the signal line SGL(2) and the third switching element TrS corresponding to the signal line SGL(8).

The control circuit 122 (refer to FIG. 1) sequentially supplies the selection signal ASW to the selection signal lines Lsel. Through the operations of the third switching elements TrS, the signal line selection circuit 16 sequentially selects the signal lines SGL in one of the signal line blocks in a time-division manner. The signal line selection circuit 16 selects one of the signal lines SGL in each of the signal line blocks. With the above-described configuration, the detection device 1 can reduce the number of integrated circuits (ICs) including the detection circuit 48 or the number of terminals of the ICs.

The signal line selection circuit 16 may couple more than one signal line SGL to the detection circuit 48 collectively. Specifically, the control circuit 122 (refer to FIG. 1) simultaneously supplies the selection signal ASW to the selection signal lines Lsel. With this operation, the signal line selection circuit 16 selects, by the operations of the third switching elements TrS, the signal lines SGL (for example, six signal lines SGL) in one of the signal line blocks, and couples the signal lines SGL to the detection circuit 48. As a result, signals detected in each group area PAG are output to the detection circuit 48. In this case, signals from the partial detection areas PAA (photodiodes PD) in each group area PAG are put together and output to the detection circuit 48.

By the operations of the gate line drive circuit 15 and the signal line selection circuit 16, the detection is performed for each group area PAG. As a result, the intensity of the detection signal Vdet obtained by one time of detection increases, so that the sensor sensitivity can be improved. In addition, time required for the detection can be reduced. Consequently, the detection device 1 can repeatedly perform the detection in a short time, and thus, can improve a signal-to-noise (S/N) ratio, and can accurately detect a change in the biological information with time, such as the pulse wave.

As illustrated in FIG. 3, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 4) included in each of the partial detection areas PAA.

As illustrated in FIG. 4, each of the partial detection areas PAA includes the photodiode PD, the capacitive element Ca, and the first switching element Tr. FIG. 4 illustrates two of the gate lines GCL(m) and GCL(m+1) arranged in the second direction Dy among the gate lines GCL and illustrates two signal lines SGL(n) and SGL(n+1) arranged in the first direction Dx among the signal lines SGL. The partial detection area PAA is an area surrounded by the gate lines GCL and the signal lines SGL. Each of the first switching elements Tr is provided correspondingly to each of the photodiodes PD. The first switching element Tr includes a thin-film transistor, and in this example, includes an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the photodiode PD and the capacitive element Ca.

The anode of the photodiode PD is supplied with the sensor power supply signal VDDSNS from the power supply circuit 123. The signal line SGL and the capacitive element Ca are supplied with the reference signal COM that serves as an initial potential of the signal line SGL and the capacitive element Ca from the power supply circuit 123.

When the partial detection area PAA is irradiated with light, a current corresponding to an amount of light flows through the photodiode PD. As a result, an electrical charge is stored in the capacitive element Ca. After the first switching element Tr is turned on, a current corresponding to the electrical charge stored in the capacitive element Ca flows through the signal line SGL. The signal line SGL is coupled to the detection circuit 48 through a corresponding one of the third switching elements TrS of the signal line selection circuit 16. Thus, the detection device 1 can detect a signal corresponding to the amount of the light irradiating the photodiode PD in each of the partial detection areas PAA or signals corresponding to the amounts of the light irradiating the photodiodes PD in each group area PAG.

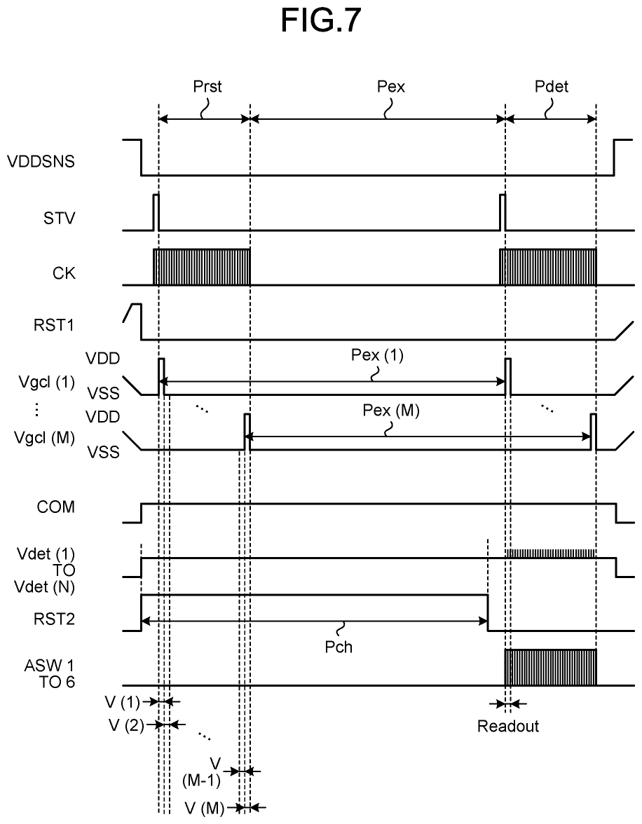

During a reading period Pdet (refer to FIG. 7), a switch SSW of the detection circuit 48 is turned on, and the detection circuit 48 is coupled to the signal lines SGL. The detection signal amplifier 42 of the detection circuit 48 converts a variation of a current supplied from the signal lines SGL into a variation of a voltage, and amplifies the result. A reference potential (Vref) having a fixed potential is supplied to a non-inverting input portion (+) of the detection signal amplifier 42, and the signal lines SGL are coupled to an inverting input portion (−) of the detection signal amplifier 42. In the present embodiment, the same signal as the reference signal COM is supplied as a reference potential (Vref) voltage. The detection signal amplifier 42 includes a capacitive element Cb and a reset switch RSW. During a reset period Prst (refer to FIG. 7), the reset switch RSW is turned on, and an electrical charge of the capacitive element Cb is reset.

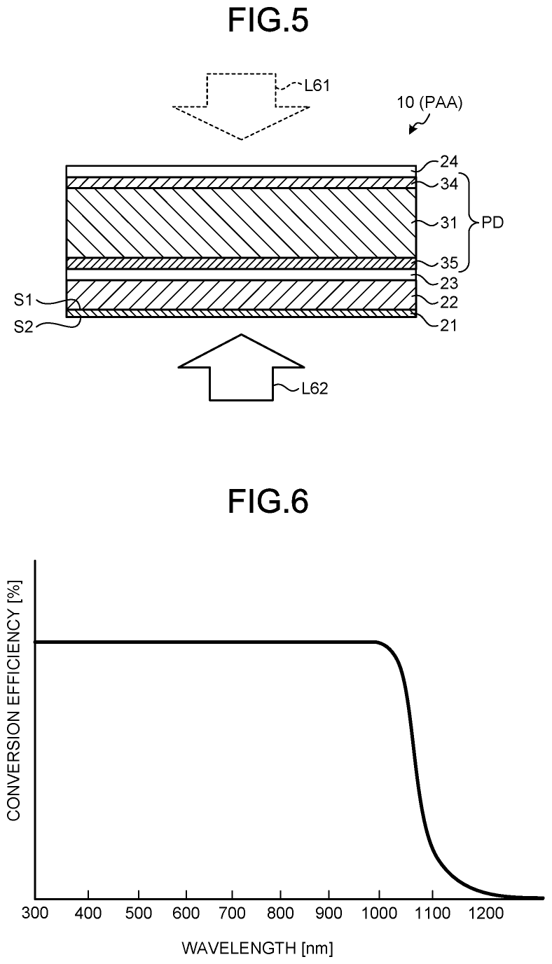

The following describes a configuration of the photodiode PD. FIG. 5 is a sectional view illustrating a schematic sectional configuration of the sensor. FIG. 6 is a graph schematically illustrating a relation between the wavelength and a conversion efficiency of light incident on the photodiode.

As illustrated in FIG. 5, the sensor 10 includes the sensor base member 21, a TFT layer 22, an insulating layer 23, the photodiode PD, and a protection film 24. The sensor base member 21 is an insulating base member and is made using, for example, glass or resin material. The sensor base member 21 is not limited to having a flat plate shape, and may have a curved surface. In this case, the sensor base member 21 may be formed of a film-shaped resin. The sensor base member 21 has a first surface S1 and a second surface S2 on the opposite side of the first surface S1. The TFT layer 22, the insulating layer 23, the photodiode PD, and the protection film 24 are stacked on the first surface S1 in the order as listed.

The TFT layer 22 is used for circuits such as the gate line drive circuit 15 and the signal line selection circuit 16 described above. The TFT layer 22 is also provided with thin-film transistors (TFTs), such as the first switching element Tr, and various types of wiring, such as the gate lines GCL and the signal lines SGL. The sensor base member 21 and the TFT layer 22, which serve as a drive circuit board that drives the sensor for each predetermined detection area, are also called a backplane.

The insulating layer 23 is an inorganic insulating layer. For example, an oxide such as silicon oxide (SiO₂) or a nitride such as silicon nitride (SiN) is used as the insulating layer 23.

The photodiode PD is provided on the insulating layer 23. The photodiode PD includes a photoelectric conversion layer 31, a cathode electrode 35, and an anode electrode 34. The cathode electrode 35, the photoelectric conversion layer 31, and the anode electrode 34 are stacked in the order as listed, in a direction orthogonal to the first surface S1 of the sensor base member 21. The stacking order in the photodiode PD may be as follows: the anode electrode 34, the photoelectric conversion layer 31, and the cathode electrode 35.

Characteristics (such as a voltage-current characteristic and a resistance value) of the photoelectric conversion layer 31 vary depending on the irradiated light. An organic material is used as the material of the photoelectric conversion layer 31. Specifically, a low-molecular organic material such as C₆₀ (fullerene), phenyl-C₆₁-butyric acid methyl ester (PCBM), copper phthalocyanine (CuPc), fluorinated copper phthalocyanine (F₁₆CuPc), rubrene (5,6,11,12-tetraphenyltetracene), or PDI (derivative of perylene) can be used as the photoelectric conversion layer 31.

The photoelectric conversion layer 31 can be formed by a vapor deposition method (dry process) using any of the above-listed low-molecular organic materials. In this case, the photoelectric conversion layer 31 may be a laminated film of CuPc and F₁₆CuPc, or a laminated film of rubrene and C₆₀. The photoelectric conversion layer 31 can also be formed by an application method (wet process). In this case, a material obtained by combining any of the above-listed low-molecular organic materials with a polymeric organic material is used as the photoelectric conversion layer 31. For example, poly(3-hexylthiophene) (P3HT) or F8-alt-benzothiadiazole (F8BT) can be used as the polymeric organic material. The photoelectric conversion layer 31 can be a film in a state of a mixture of P3HT and PCBM, or a film in a state of a mixture of F8BT and PDI.

The cathode electrode 35 faces the anode electrode 34 with the photoelectric conversion layer 31 interposed therebetween. A light-transmitting conductive material such as indium tin oxide (ITO) is used as the anode electrode 34. A metal material such as silver (Ag) or aluminum (Al) is used as the cathode electrode 35. Alternatively, the cathode electrode 35 may be an alloy material containing at least one or more of these metal materials.

The cathode electrode 35 can be formed as a light-transmitting transflective electrode by controlling the film thickness of the cathode electrode 35. For example, the cathode electrode 35 is formed of an Ag thin film having a film thickness of 10 nm so as to have light transmittance of approximately 60%. In this case, the photodiode PD can detect light emitted from both surface sides of the sensor base member 21, for example, both the first light L61 emitted from the first surface S1 side and the second light L62 emitted from the second surface S2 side.

The protection film 24 is provided so as to cover the anode electrode 34. The protection film 24 is a passivation film and is provided to protect the photodiode PD.

The horizontal axis of the graph illustrated in FIG. 6 represents the wavelength of the light incident on the photodiode PD, and the vertical axis of the graph represents an external quantum efficiency of the photodiode PD. The external quantum efficiency is expressed as a ratio between the number of photons of the light incident on the photodiode PD and a current that flows from the photodiode PD to the external detection circuit 48.

As illustrated in FIG. 6, the photodiode PD has an excellent efficiency in a wavelength range from approximately 300 nm to approximately 1000 nm. That is, the photodiode PD has a sensitivity for wavelengths of both the first light L61 emitted from the first light sources 61 and the second light L62 emitted from the second light sources 62. Therefore, each of the photodiodes PD can detect a plurality of beams of light having different wavelengths.

The following describes an operation example of the detection device 1. FIG. 7 is a timing waveform diagram illustrating the operation example of the detection device. As illustrated in FIG. 7, the detection device 1 has the reset period Prst, an effective exposure period Pex, and the reading period Pdet. The power supply circuit 123 supplies the sensor power supply signal VDDSNS to the anode of the photodiode PD over the reset period Prst, the effective exposure period Pex, and the reading period Pdet. The sensor power supply signal VDDSNS is a signal for applying a reverse bias between the anode and the cathode of the photodiode PD. For example, the reference signal COM of substantially 0.75 V is applied to the cathode of the photodiode PD, and the sensor power supply signal VDDSNS of substantially −1.25 V is applied to the anode of the photodiode PD. As a result, a reverse bias of substantially 2.0 V is applied between the anode and the cathode. At the time of detection of a wavelength of 850 nm, the reverse bias of 2 V is applied to the photodiode PD so as to obtain a high sensitivity of 0.5 A/W to 0.7 A/W, preferably approximately 0.57 A/W. The following characteristics of the photodiode are used: the dark current density is 1.0×10⁻⁷ A/cm² when the reverse bias of 2 V is applied, and the photocurrent density is 1.2×10⁻³ A/cm² when light having an output of substantially 2.9 mW/cm² and a wavelength of 850 nm is detected. The external quantum efficiency (EQE) is approximately 1.0 when the reverse bias of 2 V is applied at the time when the photodiode is irradiated with the light having a wavelength of 850 nm. The control circuit 122 sets the reset signal RST2 to “H”, and then, supplies the start signal STV and the clock signal CK to the gate line drive circuit 15 to start the reset period Prst. During the reset period Prst, the control circuit 122 supplies the reference signal COM to the reset circuit 17, and uses the reset signal RST2 to turn on the fourth switching elements TrR for supplying a reset voltage. This operation supplies the reference signals COM as the reset voltage to the signal lines SGL. The reference signal COM is set to, for example, 0.75 V.

During the reset period Prst, the gate line drive circuit 15 sequentially selects each of the gate lines GCL based on the start signal STV, the clock signal CK, and the reset signal RST1. The gate line drive circuit 15 sequentially supplies the gate drive signals Vgcl {Vgcl(1) to Vgcl(M)} to the gate lines GCL. The gate drive signal Vgcl has a pulsed waveform having a power supply voltage VDD serving as a high-level voltage and a power supply voltage VSS serving as a low-level voltage. In FIG. 7, M gate lines GCL (where M is, for example, 256) are provided, and the gate drive signals Vgcl(1) . . . , Vgcl(M) are sequentially supplied to the respective gate lines GCL. Thus, the first switching elements Tr are sequentially brought into a conducting state and supplied with the reset voltage on a row-by-row basis. For example, a voltage of 0.75 V of the reference signal COM is supplied as the reset voltage.

Thus, during the reset period Prst, the capacitive elements Ca of all the partial detection areas PAA are sequentially electrically coupled to the signal lines SGL, and are supplied with the reference signal COM. As a result, the electrical charges stored in the capacitance of the capacitive elements Ca are reset. The capacitance of the capacitive elements Ca of some of the partial detection areas PAA can be reset by partially selecting the gate lines and the signal lines SGL.

Examples of the exposure timing control method include a control method of exposure during scanning time of gate line and a full-time control method of exposure. In the control method of exposure during scanning time of gate line, the gate drive signals {Vgcl(1) to Vgcl(M)} are sequentially supplied to all the gate lines GCL coupled to the photodiodes PD serving as the detection targets, and all the photodiodes PD serving as the detection targets are supplied with the reset voltage. Then, after all the gate lines GCL coupled to the photodiodes PD serving as the detection targets are set to a low voltage (the first switching elements Tr are turned off), the exposure starts, whereby the exposure is performed during the effective exposure period Pex. After the exposure ends, the gate drive signals {Vgcl(1) to Vgcl(M)} are sequentially supplied to the gate lines GCL coupled to the photodiodes PD serving as the detection targets as described above and reading is performed during the reading period Pdet. In the full-time control method of exposure, control for performing the exposure can also be performed during the reset period Prst and the reading period Pdet (full-time exposure control). In this case, the effective exposure period Pex(1) starts after the gate drive signal Vgcl(M) is supplied to the gate line GCL. The term “effective exposure periods Pex{(1), . . . , (M)}” refers to a period during which the capacitive elements Ca are charged from the photodiodes PD. The start timing and the end timing of the actual effective exposure periods Pex(1), . . . , Pex(M) are different among the partial detection areas PAA corresponding to the gate lines GCL. Each of the effective exposure periods Pex(1), . . . , Pex(M) starts when the gate drive signal Vgcl changes from the power supply voltage VDD serving as the high-level voltage to the power supply voltage VSS serving as the low-level voltage during the reset period Prst. Each of the effective exposure periods Pex(1), . . . , Pex(M) ends when the gate drive signal Vgcl changes from the power supply voltage VSS to the power supply voltage VDD during the reading period Pdet. The lengths of the exposure time of the effective exposure periods Pex(1), . . . , Pex(M) are equal.

In the control method of exposure during scanning time of gate line, a current flows corresponding to the light irradiating the photodiode PD in each of the partial detection areas PAA during the effective exposure periods Pex{(1), . . . , (M)}. As a result, an electrical charge is stored in each of the capacitive elements Ca.

At a time before the reading period Pdet starts, the control circuit 122 sets the reset signal RST2 to a low-level voltage. This operation stops operation of the reset circuit 17. The reset signal may be set to a high-level voltage only during the reset period Prst. During the reading period Pdet, the gate line drive circuit 15 sequentially supplies the gate drive signals Vgcl(1) . . . , Vgcl(M) to the gate lines GCL in the same manner as during the reset period Prst.

Specifically, the gate line drive circuit 15 supplies the gate drive signal Vgcl(1) at the high-level voltage (power supply voltage VDD) to the gate line GCL(1) during a period V(1). The control circuit 122 sequentially supplies the selection signals ASW1, . . . , ASW6 to the signal line selection circuit 16 during a period in which the gate drive signal Vgcl(1) is at the high-level voltage (power supply voltage VDD). This operation sequentially or simultaneously couples the signal lines SGL of the partial detection areas PAA selected by the gate drive signal Vgcl(1) to the detection circuit 48. As a result, the detection signal Vdet for each of the partial detection areas PAA is supplied to the detection circuit 48. A time of, for example, approximately 20 μs (substantially 20 μs) elapses from when the gate drive signal Vgcl(1) is set to the high level to when the first selection signal ASW1 starts to be supplied, and a time of, for example, approximately 60 μs (substantially 60 μs) elapses while each of the selection signals ASW1, . . . , ASW6 is supplied. Such a high-speed response can be achieved by using thin-film transistors (TFTs) made using low-temperature polysilicon (LTPS) having mobility of substantially 40 cm²/Vs.

In the same manner, the gate line drive circuit 15 supplies the gate drive signals Vgcl(2), . . . , Vgcl(M−1), Vgcl(M) at the high-level voltage to gate lines GCL(2), . . . , GCL(M−1), GCL(M) during periods V(2), . . . , V(M−1), V(M), respectively. That is, the gate line drive circuit 15 supplies the gate drive signal Vgcl to the gate line GCL during each of the periods V(1), V(2), . . . , V(M−1), V(M). The signal line selection circuit 16 sequentially selects each of the signal lines SGL based on the selection signal ASW in each period in which the gate drive signal Vgcl is set to the high-level voltage. The signal line selection circuit 16 sequentially couples each of the signal lines SGL to one detection circuit 48. Thus, the detection device 1 can output the detection signals Vdet of all the partial detection areas PAA to the detection circuit 48 during the reading period Pdet.

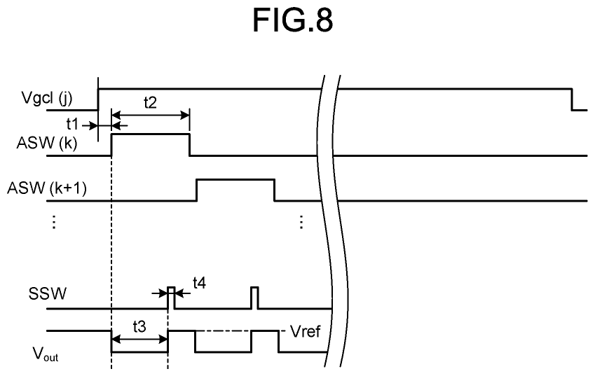

FIG. 8 is a timing waveform diagram illustrating an operation example during a drive period of one of the gate lines included in a reading period Readout in FIG. 7. With reference to FIG. 8, the following describes the operation example during the supply period Readout of one of the gate drive signals Vgcl(j) in FIG. 7. In FIG. 7, the reference numeral of the supply period “Readout” is assigned to the first gate drive signal Vgcl(1), but the same applies to the other gate drive signals Vgcl(2) . . . , Vgcl(M). The index j is any one of the natural numbers 1 to M.

As illustrated in FIGS. 8 and 4, an output (Vₒᵤₜ) of each of the third switching elements TrS has been reset to the reference potential (Vref) voltage in advance. The reference potential (Vref) voltage serves as a reset voltage, and is set to, for example, 0.75 V. Then, the gate drive signal Vgcl(j) is set to a high level, and the first switching elements Tr of a corresponding row are turned on. Thus, each of the signal lines SGL of each row is set to a voltage corresponding to the electrical charge stored in the capacitor (capacitive element Ca) of the partial detection area PAA. After a period t1 elapses from a rise of the gate drive signal Vgcl(j), a period t2 starts in which the selection signal ASW(k) is set to a high level. After the selection signal ASW(k) is set to the high level and the third switching element TrS is turned on, the output (Vₒᵤₜ) of the third switching element TrS (refer to FIG. 4) is changed to a voltage corresponding to the electrical charge stored in the capacitor (capacitive element Ca) of the partial detection area PAA coupled to the detection circuit 48 through the third switching element TrS, by the electrical charge stored in the capacitor (capacitive element Ca) of the partial detection area PAA (period t3). In the example of FIG. 8, this voltage is reduced from the reset voltage as illustrated in the period t3. Then, after the switch SSW is turned on (high-level period t4 of an SSW signal), the electrical charge stored in the capacitor (capacitive element Ca) of the partial detection area PAA moves to a capacitor (capacitive element Cb) of the detection signal amplifier 42 of the detection circuit 48, and the output voltage of the detection signal amplifier 42 is set to a voltage corresponding to the electrical charge stored in the capacitive element Cb. At this time, the potential of an inverting input portion of the detection signal amplifier 42 is set to an imaginary short-circuit potential of the operational amplifier, and therefore, returns to the reference potential (Vref). The A/D converter 43 reads the output voltage of the detection signal amplifier 42. In the example of FIG. 8, waveforms of the selection signals ASW(k), ASW(k+1), . . . corresponding to the signal lines SGL of the respective columns are set to be a high level to sequentially turn on the third switching elements TrS, and the same operation is sequentially performed. This operation sequentially reads the electrical charges stored in the capacitors (capacitive elements Ca) of the partial detection areas PAA coupled to the gate line GCL. ASW(k), ASW(k+1), . . . in FIG. 8 are, for example, any of ASW 1 to 6 in FIG. 7.

Specifically, after the period t4 starts in which the switch SSW is on, the electrical charge moves from the capacitor (capacitive element Ca) of the partial detection area PAA to the capacitor (capacitive element Cb) of the detection signal amplifier 42 of the detection circuit 48. At this time, the non-inverting input (+) of the detection signal amplifier 42 is biased to the reference potential (Vref) voltage (for example, 0.75 [V]). As a result, the output (Vₒᵤₜ) of the third switching element TrS is also set to the reference potential (Vref) voltage due to the imaginary short-circuit between input ends of the detection signal amplifier 42. The voltage of the capacitive element Cb is set to a voltage corresponding to the electrical charge stored in the capacitor (capacitive element Ca) of the partial detection area PAA at a location where the third switching element TrS is turned on in response to the selection signal ASW(k). After the output (Vₒᵤₜ) of the third switching element TrS is set to the reference potential (Vref) voltage due to the imaginary short-circuit, the output of the detection signal amplifier 42 reaches a capacitance corresponding to the voltage of the capacitive element Cb, and this output voltage is read by the A/D converter 43. The voltage of the capacitive element Cb is, for example, a voltage between two electrodes in a capacitor constituting the capacitive element Cb.

The period t1 is, for example, 20 [μs]. The period t2 is, for example, 60 [μs]. The period t3 is, for example, 44.7 [μs]. The period t4 is, for example, 0.98 [μs].

Although FIGS. 7 and 8 illustrate the example in which the gate line drive circuit 15 selects the gate line GCL individually, the number of the gate lines GCL to be selected is not limited to this example. The gate line drive circuit 15 may simultaneously select a predetermined number (two or more) of the gate lines GCL and sequentially supply the gate drive signals Vgcl to the gate lines GCL in units of the predetermined number of the gate lines GCL. The signal line selection circuit 16 may also simultaneously couple a predetermined number (two or more) of the signal lines SGL to one detection circuit 48. Moreover, the gate line drive circuit 15 may skip some of the gate lines GCL and scan the remaining ones. The dynamic range is, for example, approximately 10³ when the effective exposure period Pex is approximately 4.3 ms. A high resolution can be achieved by setting the frame rate to approximately 4.4 fps (substantially 4.4 fps).

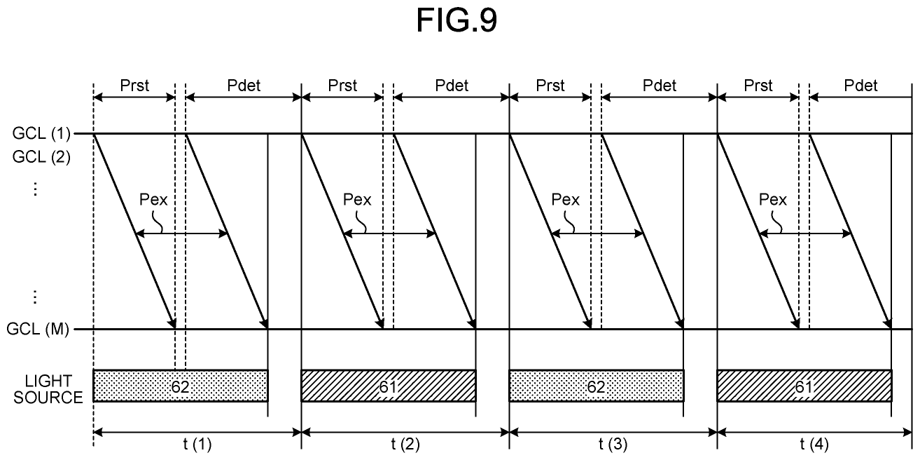

The following describes an operation example of the sensor 10, the first light sources 61, and the second light sources 62. FIG. 9 is an explanatory diagram for explaining a relation between driving of the sensor and lighting operations of the light sources in the detection device.

As illustrated in FIG. 9, during each of the periods t(1) to t(4), the detection device 1 performs the processing in the reset period Prst, the effective exposure period Pex{(1), . . . , (M)}, and the reading period Pdet described above. During the reset period Prst and the reading period Pdet, the gate line drive circuit 15 sequentially performs scanning from the gate line GCL(1) to gate line GCL(M).

During the period t(1), the second light sources 62 are on, and the first light sources 61 are off. As a result, in the detection device 1, currents flow from the photodiodes PD through the signal lines SGL to the detection circuit 48 based on the second light L62 emitted from the second light sources 62. During the period t(2), the first light sources 61 are on, and the second light sources 62 are off. As a result, in the detection device 1, currents flow from the photodiodes PD through the signal lines SGL to the detection circuit 48 based on the first light L61 emitted from the first light sources 61. In the same manner, during the period t(3), the second light sources 62 are on, and the first light sources 61 are off; and during the period t(4), the first light sources 61 are on, and the second light sources 62 are off.

In this manner, the first light sources 61 and the second light sources 62 are caused to be on in a time-division manner at intervals of the period t. This operation outputs the first detection signals detected by the photodiodes PD based on the first light L61 and the second detection signals detected by the photodiodes PD based on the second light L62 to the detection circuit 48 in a time-division manner. Consequently, the first detection signals and the second detection signals are restrained from being output to the detection circuit 48 in a mutually superimposed manner. As a result, the detection device 1 can well detect the various types of the biological information.

The driving method of the first light sources 61 and the second light sources 62 can be changed as appropriate. For example, in FIG. 9, the first light sources 61 and the second light sources 62 are alternately caused to be on at intervals of the period t. However, the driving method is not limited thereto. The first light sources 61 may be turned on in successive periods t, and then, the second light sources 62 may be turned on in successive periods t. The first light sources 61 and the second light sources 62 may be simultaneously turned on in each period t. FIG. 9 illustrates an example of the full-time control method of exposure. However, also in the control method of exposure during scanning time of gate line, the first light sources 61 and the second light sources 62 may be alternately driven at intervals of the period t in the same manner as illustrated in FIG. 9.

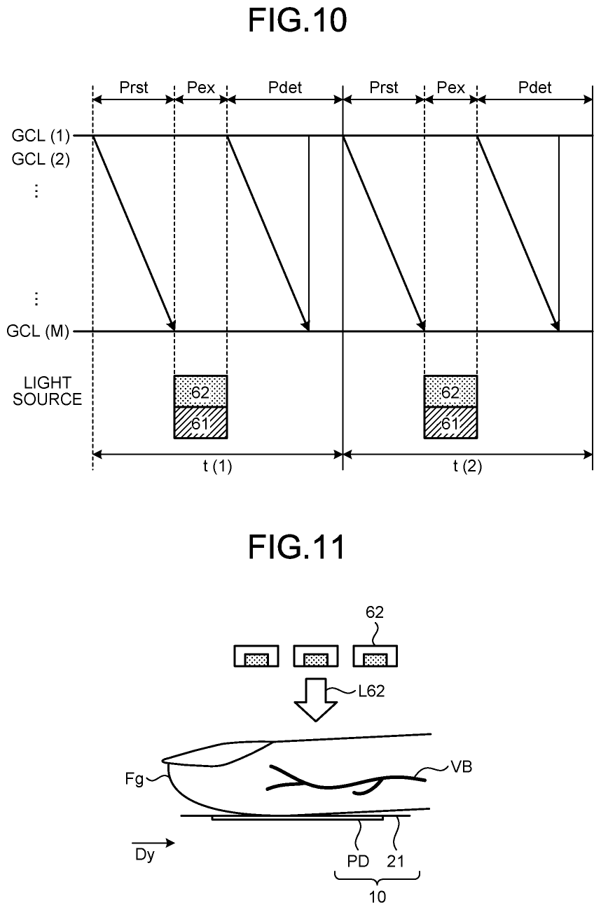

FIG. 10 is an explanatory diagram for explaining a relation between the driving of the sensor and the lighting operations of the light sources different from the relation of FIG. 9. In the example illustrated in FIG. 10, the first light sources 61 and the second light sources 62 are on during the effective exposure period Pex, and are off during the reset period Prst and the reading period Pdet. Through these operations, the detection device 1 can reduce power consumption required for the detection.

The lighting operations are not limited to the example illustrated in FIG. 10. The first light sources 61 and the second light sources 62 may be continuously turned on over all the periods including the reset period Prst, the effective exposure period Pex, and the reading period Pdet. Either the first light sources 61 or the second light sources 62 may be turned on during the effective exposure period Pex, and the first light sources 61 and the second light sources 62 may be alternately on at intervals of the period t.

FIG. 11 is a schematic view illustrating an exemplary positional relation between the second light sources 62, the sensor 10, and a blood vessel VB in the finger Fg. The second light L62 emitted from the second light sources 62 (at least one or more of second light sources 62-1, 62-2, and 62-3) is transmitted through the finger Fg and enters the photodiode PD of each of the partial detection areas PAA. At this time, the transmittance of the second light L62 through the finger Fg changes in accordance with pulsation of the blood vessel VB in the finger Fg. Therefore, the pulse wave can be detected based on the periods of the variation (amplitude) of the detection signal Vdet during a period of time longer than or equal to the pulsation period of the blood vessel VB.

In the case of detecting the pulse wave, the second light sources 62 preferably emit infrared light. Specifically, as described above, the second light L62 may have a wavelength in a range from 780 nm to 900 nm, for example, at approximately 850 nm, or may have a wavelength in a range from 800 nm to 930 nm. In the case of detecting the pulse wave, the wavelength of the second light L62 from the second light sources 62 only needs to be in a range from 500 nm to 950 nm.

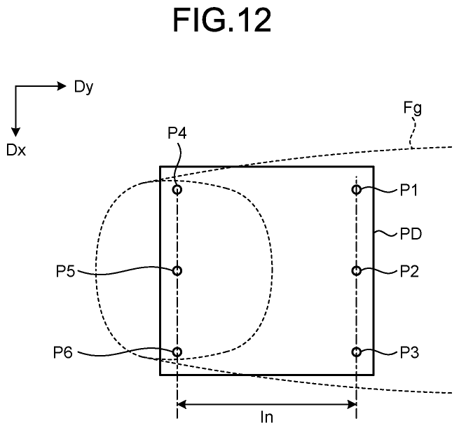

FIG. 12 is a schematic view illustrating positions of a plurality of partial detection points (points P1, P2, P3, P4, P5, and P6) in the photodiode PD that are exemplarily set when the planar detection area AA formed by the photodiodes PD provided so as to face the finger Fg is viewed in the plan view. As exemplified by the points P1, P2, P3, P4, P5, and P6 in FIG. 12, when the pulse wave is detected at each of the points the positions of which are different from one another, a gap corresponding to a distance between the points is generated between the pulse waves detected at the respective points. Using this phenomenon, a pulse wave velocity can be calculated based on a relation between the distance between two different points and the temporal shift between the pulse waves detected at the two points. Specifically, as illustrated in FIG. 11, the blood vessel has a three-dimensionally curved shape, and the vascular pattern having the three-dimensionally curved shape is detected by the sensors (partial detection areas PAA) arranged in a matrix having a row-column configuration as illustrated in FIG. 3. Since the blood vessel on a body surface does not vary significantly in the depth direction, a detected two-dimensional vascular pattern may be used as an approximate pattern of the three-dimensional vascular pattern, or the three-dimensional vascular pattern may be obtained by performing an image analysis on the detected two-dimensional vascular pattern. The pulse wave velocity is calculated based on the relation between the length of the blood vessel between two different points on the detected vascular pattern and the temporal shift. For example, when the pulse wave is observed at the point P2 and the point P5 in FIG. 12, and the points P2 and P5 are located on the vascular pattern, the pulse wave generally propagates from a position closer to the heart to a position farther therefrom, and therefore, propagates from the point P2 to the point P5. In this case, the pulse wave velocity can be calculated based on a distance In between the points P2 and P5 and the temporal shift between the pulse waves at the points P2 and P5. That is, the temporal shift between the pulse wave at the point P2 and the pulse wave at the point P5 corresponds to a time spent for propagation of the pulse wave between the two points at a distance of the distance In from each other.

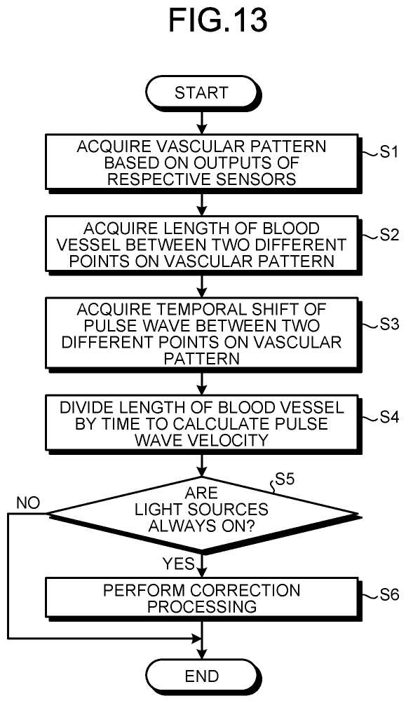

FIG. 13 is a flowchart illustrating an exemplary flow of processing for correcting the temporal shift that branches in accordance with a control mode of a lighting time of the light sources. For example, the output processor 50 performs such processing. First, the pattern (vascular pattern) of the blood vessel VB in a living body tissue (refer to FIG. 11) facing the detection area AA is acquired based on the outputs of the respective sensors included in the detection area AA, that is, the outputs of the respective photodiodes PD of the partial detection areas PAA (Step S1). Then, the length of the blood vessel between two different points (for example, the points P2 and P5, refer to FIG. 12) on the vascular pattern is acquired (Step S2). Then, the temporal shift of the pulse wave between the two different points (for example, the points P2 and P5, refer to FIG. 12) on the vascular pattern is acquired (Step S3). The temporal shift of the pulse wave herein refers to “shift time” to be described later. Then, the length of the blood vessel between the two different points (for example, the points P2 and P5, refer to FIG. 12) on the vascular pattern is divided by time (shift time) to calculate the pulse wave velocity (Step S4). The length of the blood vessel is calculated based on the detected vascular pattern and the distance between the two different points (for example, the points P2 and P5, refer to FIG. 12) on the vascular pattern. For example, the length of the blood vessel between the two different points on the detected vascular pattern is obtained by image analysis on the detected two-dimensional vascular pattern or the three-dimensional vascular pattern. If, as described with reference to FIG. 9, the light sources (for example, the light sources 62) that emit the light for detecting the vascular pattern and the pulse wave are in an operation mode of being always left turned on (Yes at Step S5), correction processing for correcting the temporal shifts of the effective exposure periods Pex{(1), . . . , (M)} (to be described later) is performed (Step S6). In contrast, if, as described with reference to FIG. 10, the light sources are not in the operation mode of being always left turned on (No at Step S5), the correction processing of Step S6 is not performed. The measuring device may be a device in which the light sources have only a full-time light-on control system, or may be a device in which the light sources have only a control system of exposure during scanning time of gate line. In the device in which the light sources have only the full-time light-on control system, the processing at Step S5 of FIG. 13 is not performed. In the device in which the light sources have only the control system of exposure during scanning time of gate line, the branch from Step S5 to “No” in FIG. 13 is skipped.

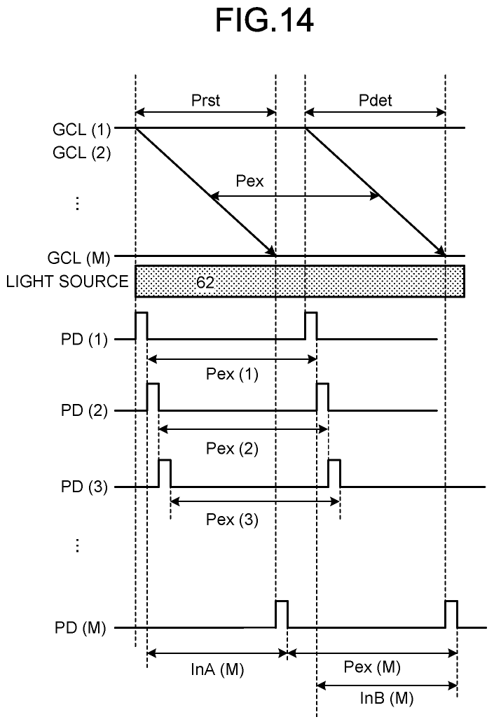

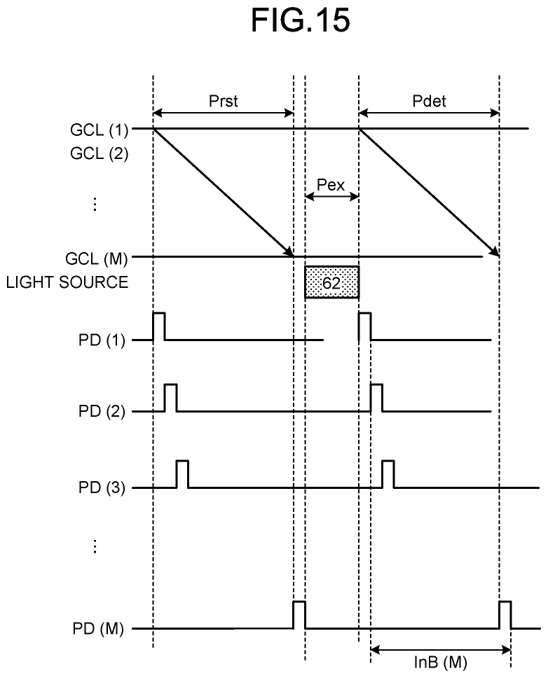

As described with reference to FIGS. 7, 9, and 10, the temporal shifts of output timings occur between the partial detection areas PAA that are arranged in the second direction Dy and differ from one another in supply timing of the gate drive signal Vgcl. As described with reference to FIGS. 7 and 9, when the reset period Prst and the reading period Pdet overlap the lighting period of the light sources, the temporal shifts of the effective exposure period Pex occur among the partial detection areas PAA that are arranged in the second direction Dy and differ from one another in supply timing of the gate drive signal Vgcl. The following describes such temporal shifts with reference to FIGS. 14 and 15.

FIG. 14 is a timing diagram for explaining the temporal shifts of the effective exposure periods Pex{(1), . . . , (M)} and the output timings when the reset period Prst and the reading period Pdet overlap the lighting period of the second light sources 62. In FIG. 14 and FIG. 15 (to be described later), different values are given in parentheses of the gate lines GCL and the photodiodes PD having different supply timing of the gate drive signal Vgcl. For example, the photodiode PD(1) is coupled, through the first switching element Tr, to the gate line GCL(1) that is first supplied with the gate drive signal Vgcl during the reset period Prst; and the photodiode PD(M) is coupled, through the first switching element Tr, to the gate line GCL(M) that is last supplied with the gate drive signal Vgcl during the reset period Prst. The gate drive signal Vgcl is supplied in the order of the gate line GCL(1), the gate line GCL(2), . . . , the gate line GCL(M).

As illustrated in FIG. 14, during the reset period Prst, the gate drive signal Vgcl is supplied to the gate lines GCL, such as the gate lines GCL(1), GCL(2), . . . , GCL(M), arranged in the second direction Dy at different timings from one another, and as a result, the temporal shifts of the reset timings occur between the photodiode PD(1), the photodiode PD(2), . . . , the photodiode PD(M). The reset of the photodiode PD refers to reset of the capacitance of the capacitive element Ca of the partial detection area PAA provided with the photodiode PD.

In the reset period Prst illustrated in FIG. 14, a rise of a pulse of the gate drive signal Vgcl supplied to each of the photodiodes PD(1), PD(2), . . . , PD(M) is defined as start timing of the reset, and a fall of the pulse is defined as completion timing of the reset. Then, the temporal shift of the completion timing of the reset can be represented by a shift of timing of the fall of the pulse. The degree of temporal shift of the completion timing of the reset is maximized between the photodiode PD(1) and the photodiode PD(M). FIG. 14 illustrates such maximum temporal shift of the completion timing of the reset as time InA(M).

When the reset period Prst and the reading period Pdet overlap the lighting period of the light sources as illustrated in FIGS. 9 and 14, each of the effective exposure periods Pex{(1), . . . , (M)} of the respective photodiodes PD starts in response to completion of the reset of a corresponding one of the photodiodes PD. Thus, the temporal shifts of the start timings occur between the effective exposure periods Pex{(1), . . . , (M)} of the photodiodes PD(1), PD(2), . . . , PD(M), respectively, due to the temporal shift of the completion timing of the reset. The term “effective exposure periods Pex{(1), . . . , (M)}” refers to the period during which the capacitive elements Ca are charged from the photodiodes PD. Each of the effective exposure periods Pex{(1), . . . , (M)} of the respective photodiodes PD ends in response to start of the reading period Pdet of a corresponding one of the photodiodes PD. Therefore, during the reading period Pdet, the gate drive signal Vgcl is supplied to the gate lines GCL, such as the gate lines GCL(1), GCL(2), . . . , GCL(M), arranged in the second direction Dy at different timings from one another, and as a result, the temporal shifts of the end timings occur between the effective exposure periods Pex{(1), . . . , (M)} of the photodiodes PD(1), PD(2), . . . , PD(M), respectively.

As described above, when the reset period Prst and the reading period Pdet overlap the lighting period of the light sources as illustrated in FIGS. 9 and 14, the gate drive signal Vgcl is supplied to the gate lines GCL, such as the gate lines GCL(1), GCL(2), . . . , GCL(M), arranged in the second direction Dy at different timings from one another, and as a result, the temporal shifts of the start timing and the end timing occur between the effective exposure periods Pex{(1), . . . , (M)} of the photodiodes PD(1), PD(2), . . . , PD(M), respectively. FIG. 14 illustrates the effective exposure periods Pex of the photodiodes PD(1), PD(2), . . . , PD(M) as Pex(1), Pex(2), . . . , Pex(M). In this manner, the fact that the temporal shifts occur in the effective exposure periods Pex{(1), . . . , (M)} of the photodiodes PD(1), PD(2), . . . , PD(M), respectively, indicates that, when the pulsation is detected by each of the photodiodes PD(1), PD(2), . . . , PD(M), the timing of the pulsation detected thereby includes the temporal shift of a corresponding one of the effective exposure periods Pex{(1), . . . , (M)}.

During the reading period Pdet, the gate drive signal Vgcl is supplied to the gate lines GCL, such as the gate lines GCL(1), GCL(2), . . . , GCL(M), arranged in the second direction Dy at different timings from one another, and as a result, the temporal shifts of the output timings occur between the photodiodes PD(1), PD(2), . . . , PD(M). The output of the photodiode PD refers to an output based on the capacitance of the capacitive element Ca of the partial detection area PAA provided with the photodiode PD.

In the reading period Pdet, the fall of the pulse of the gate drive signal Vgcl supplied to each of the photodiodes PD(1), PD(2), . . . , PD(M) is defined as the end of a corresponding one of the effective exposure periods Pex{(1), . . . , (M)}. The rise of the pulse is defined as the start timing of the output of the photodiode PD, and the fall of the pulse is defined as the end timing of the output of the photodiode PD. When the fall of the pulse is defined as the completion timing of the output of the photodiode PD, the temporal shift of the completion timing of the output can be represented by a shift of the timing of the fall of the pulse. The degree of temporal shift of the completion timing of the output is maximized between the photodiode PD(1) and the photodiode PD(M). FIG. 14 illustrates such maximum temporal shift of the completion timing of the reset as time InB(M).

As described above, when the reset period Prst and the reading period Pdet overlap the lighting period of the light sources as illustrated in FIGS. 9 and 14, the temporal shifts occur in the supply timing of the gate drive signal Vgcl, and as a result, the temporal shifts of the effective exposure periods Pex{(1), . . . , (M)} and the output timings occur.

FIG. 15 is a timing diagram for explaining the temporal shifts of the output timings when the reset period Prst and the reading period Pdet do not overlap the lighting period of the second light sources 62. In the case of the example illustrated in FIG. 15, since the temporal shifts due to the effective exposure periods Pex do not occur, the correction of such temporal shifts is not performed. Even in the example illustrated in FIG. 15, the shift of the completion timing of the output occurs for the same reason as that described with reference to FIG. 14. However, the correction of the temporal shift related to the completion timing of the output is eliminated by acquiring data for each frame and providing a time stamp thereto. However, when the data is acquired for each line and is provided with the time stamp, the temporal shift related to the completion timing of the output is corrected.

Specifically, even in the example illustrated in FIG. 15, the temporal shift of the completion timing of the output, such as the time InB(M), occurs in the same manner as in the example illustrated in FIG. 14. That is, during the reading period Pdet, the gate drive signal Vgcl is supplied to the gate lines GCL, such as the gate lines GCL(1), GCL(2), . . . , GCL(M), arranged in the second direction Dy at different timings from one another, and as a result, the temporal shifts of the output timings occur among the photodiodes PD(1), PD(2), . . . , PD(M). In this case, when the time stamp is provided for each output timing corresponding to each of the gate lines GCL{(1), . . . , (M)}, the temporal shift related to the completion timing of the output is corrected.

When the reset period Prst and the reading period Pdet do not overlap the lighting period of the second light sources 62 as illustrated in FIGS. 10 and 15, the start timing and the end timing of the effective exposure period Pex are determined by the start timing and the end timing of the lighting of the second light sources 62. That is, when the reset period Prst and the reading period Pdet do not overlap the lighting period of the second light sources 62, the effective exposure period Pex is common to the photodiodes PD(1), PD(2), . . . , PD(M) regardless of the shift of the supply timing of the gate drive signal Vgcl during the reset period Prst and the reading period Pdet. Therefore, when the reset period Prst and the reading period Pdet do not overlap the lighting period of the second light sources 62 as illustrated in FIGS. 10 and 15, the temporal shift of the effective exposure period Pex does not occur. In other words, in the example illustrated in FIG. 15, the lighting period of the second light sources 62 directly serves as the effective exposure period Pex.

In this manner, regardless of the relation of the reset period Prst and the reading period Pdet with the lighting period of the second light sources 62, the temporal shift of the completion timing of the output, such as the time InB(M), occurs due to the shift of the supply timing of the gate drive signal Vgcl. If the time stamp is provided for each of the photodiodes PD{(1), . . . , (M)} corresponding to the gate lines GCL{(1), . . . , (M)}, when the pulse wave velocity is calculated based on the pulsation represented by the output of each of the photodiodes PD(1), PD(2), . . . , PD(M) without taking into account the temporal shift of the completion timing of the output, the calculated pulse wave velocity includes an error caused by the temporal shift of the completion timing of the output. Therefore, in this case, in calculating the pulse wave, the temporal shifts of the output timing of the photodiodes PD(1), PD(2), . . . , PD(M) are corrected based on the supply timing of the gate drive signal Vgcl to the gate lines GCL(1), GCL(2), . . . , GCL(M).

The relation of the reset period Prst and the reading period Pdet with the lighting period of the second light sources 62 influences whether the shift of the supply timing of the gate drive signal Vgcl causes the temporal shift of the effective exposure period Pex of each of the photodiodes PD(1), PD(2), . . . , PD(M). Thus, in the embodiment in which the reset period Prst and the reading period Pdet overlap the lighting period of the second light sources 62 (refer to FIGS. 9 and 14), in calculating the pulse wave, the temporal shifts of the effective exposure periods Pex{(1), . . . , (M)} of the photodiodes PD(1), PD(2), . . . , PD(M), respectively, are further corrected based on the supply timing of the gate drive signal Vgcl to the gate lines GCL(1), GCL(2), . . . , GCL(M). In contrast, in the embodiment in which the reset period Prst and the reading period Pdet do not overlap the lighting period of the second light sources 62 (refer to FIGS. 10 and 15), the temporal shift of the effective exposure period Pex does not occur, and therefore, the temporal shift of the effective exposure period Pex is not corrected.

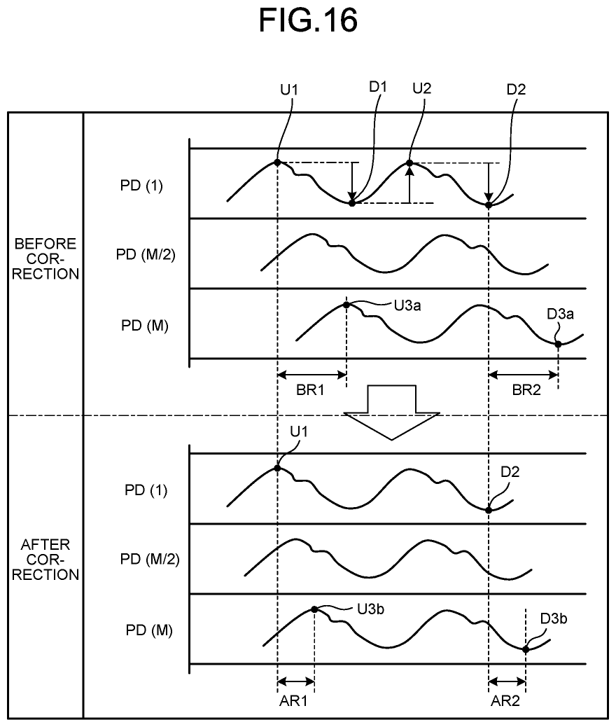

FIG. 16 is an explanatory diagram illustrating examples of the temporal shifts of the outputs from the photodiode PD(1), the photodiode PD(M/2), and the photodiode PD(M) before and after correction. As illustrated by an output waveform of PD(1) before correction in FIG. 16, the output of the photodiode PD repeats generating amplitudes, such as from a peak U1, to a bottom D1, a peak U2, a bottom D2, . . . , in response to the repeated pulses. The degree of the amplitude of the temporally output, such as the degree of continuous decrease in output value from the peak U1 to the bottom D1 or the degree of continuous increase in output value from the bottom D1 to the peak U2, is compared with a predetermined threshold of amplitude (amplitude reference value) for detecting the pulsation. For example, if the degree of the amplitude generated in a period from the peak U1 through the bottom D1 to the peak U2 is equal to or larger than the threshold, one pulse is determined to have occurred during the period. Subsequently, the relation with the pulsation is determined in the same manner for a period from the peak U2 through the bottom D2 to a peak (not illustrated) and for a period in which an amplitude of the output (not illustrated) is generated.

The threshold of amplitude is set, based on tests in advance or the like, as such a value that the amplitude of output values obtained by processing the peak U1, the bottom D1, the peak U2, and the bottom D2 illustrated in FIG. 16 into the form of the output values is handled as the amplitude of an output by the pulse wave. A specific value is determined based on, for example, a rule for processing the peak U1, the bottom D1, the peak U2, and the bottom D2 into the form of the output values by A/D conversion.

To detect and determine such amplitude of the output, the output is held on a predetermined period (for example, four seconds) basis. Although, for example, the storage 46 is used to hold such an output, the present embodiment is not limited thereto. A storage device or a storage circuit only needs to be provided that can be referred to by a component for determining the pulsation. For example, a storage for holding the output may be provided that can be used by the output processor 50.

Although a peak of the output such as the peak U1 or U2, or a bottom of the output such as the bottom D1 or D2 serves as a trigger for counting the timing of the pulsation, the present embodiment is not limited thereto. Any timing in a period in which the amplitude of the output is generated can serve as the count timing of the pulsation.

Before correction of the output, a temporal shift indicated by time BR1 occurs between the peak U1 of the output of the photodiode PD(1) and a peak U3a of the output of the photodiode PD(M). Before correction of the output, a temporal shift indicated by time BR2 occurs between the bottom D2 of the output of the photodiode PD(1) and a bottom D3a of the output of the photodiode PD(M). Each of the times BR2 and BR1 includes the temporal shift generated in accordance with the temporal shift of the supply timing of the gate drive signal Vgcl described with reference to FIGS. 14 and 15.

Thus, in the embodiment, the times BR1 and BR2 are corrected such that the temporal shift between the pulsation timing represented by the output of the photodiode PD(1) and the pulsation timing represented by the output of the photodiode PD(M) is equal to a temporal shift corresponding to the distance between the photodiode PD(1) and the photodiode PD(M). For the correction, the correction value is obtained from a relation among the scan speed of the gate lines GCL, the distance in the vascular pattern, and the angle between the extension direction at each of the positions of the vascular pattern and the scan direction. For example, if the extension direction between two points (for example, the photodiode PD(1) and the photodiode PD(M)) of the vascular pattern is the same as the scan direction (second direction Dy) of the gate lines GCL, the correction value only needs to be obtained by simply dividing the distance between the two points (of the vascular pattern) by the shift time. Herein, the term “shift time” refers to a shift time between pulse waves detected at the two points that is derived as a result of the correction of the temporal shift described above. That is, the “shift time” is a “shift time” when the same pulse wave is assumed to be observed at the two points with the “shift time” interposed therebetween while the pulse wave propagates. When the vascular pattern between the two points includes portions forming an angle with the scan direction (second direction Dy), the distance between the two points (of the vascular pattern) is further divided by the tangent of the average of the angles (tan θ).

For example, when the reset period Prst and the reading period Pdet overlap the lighting period of the second light sources 62 as described with reference to FIG. 14, the correction is performed in which the time InA(M) and the time InA(M) are subtracted from the time BR1 and the time BR2, respectively. This operation corrects the times BR1 and BR2 to times AR1 and AR2. In the time AR1, the temporal shift of the peak U3a in the output of the photodiode PD(M) with respect to the peak U1 is corrected to be a peak U3b. In the time AR2, the temporal shift of the bottom D3a in the output of the photodiode PD(M) with respect to the bottom D2 is corrected to be a bottom D3b. Such correction is merely an example and is not limited thereto. The temporal shift of the output of the photodiode PD(1) may be corrected with respect to the output of the photodiode PD(M).

When the reset period Prst and the reading period Pdet do not overlap the lighting period of the second light sources 62 as described with reference to FIG. 15, the correction is performed in which the time InB(M) is subtracted from each of the time BR1 and the time BR2. This operation corrects the times BR1 and BR2 to the times AR1 and AR2. In FIG. 16, the relation between the times before and after the correction is illustrated by the times BR1 and BR2 and the times AR1 and AR2. However, the present invention is not limited thereto. The temporal shifts of the outputs during other periods are corrected in the same manner.

When the photodiode PD(1) is provided at the point P5 (refer to FIG. 12) and the photodiode PD(M) is provided at the point P2 (refer to FIG. 12), the temporal shifts between the pulse waves generated with the distance In interposed therebetween are assumed to the times AR1 and AR2 (refer to FIG. 16). When the distance In=α [mm] and the time AR1=AR2=β [s], a pulse wave velocity γ (mm/s) in the second direction Dy between the point P5 and the point P2 can be represented as Expression (1) below.

γ=α/(1000/β) (1)

Each of the times AR1 and AR2 is a time generated by a temporal shift corresponding to the distance between the photodiode PD(1) and the photodiode PD(M). Thus, the pulse wave velocity in the second direction Dy between the photodiode PD(1) and the photodiode PD(M) can be calculated based on the relation of the distance between the photodiode PD(1) and the photodiode PD(M) with each of the times AR1 and AR2.

While the correction has been described above as an example of the relation between the photodiode PD(1) and the photodiode PD(M), the pulse wave velocity between the photodiodes PD(1), PD(2), . . . , PD(M) can be calculated by individually applying the correction using the same approach to the temporal shift of the output of each of the photodiodes PD(1), PD(2), . . . , PD(M). FIG. 16 schematically illustrates, as an example, that the outputs before and after correction of the photodiode PD(M/2) located substantially in the middle between the photodiode PD(1) and the photodiode PD(M) exhibit substantially intermediate output amplitude patterns between those of the outputs of the photodiode PD(1) and the outputs of the photodiode PD(M) before and after the correction.

The above describes the temporal shifts between the partial detection areas PAA that are arranged in the second direction Dy and differ from one another in supply timing of the gate drive signal Vgcl. In the same manner, the correction can be performed on the temporal shifts caused by the selection signals ASW (refer to FIGS. 7 and 8) being supplied to the partial detection areas PAA arranged in the first direction Dx at different timings from one another. The corrected temporal shift refers to a temporal shift of the completion timing of the output of each of the photodiodes PD. Such correction is defined by replacing the “shift of the supply timing of the gate drive signal Vgcl” in the above description of the correction with the “shift of the supply timing of the selection signal ASW”. By such correction, the pulse wave velocity between two points in the partial detection areas PAA arranged in the first direction Dx can be accurately calculated.

While the description with reference to FIG. 12 exemplarily deals with the pulse wave velocity between the point P5 and the point P2, the pulse wave velocity between other two points, such as between the point P4 and the point P1 or between the point P6 and the point P3, can be calculated in the same manner. The pulse wave velocity between two points can also be calculated for the different two points among the point P1, the point P2, and the point P3, or between different two points among the point P4, the point P5, and the point P6 by employing the above-described approach of replacing the “shift of the supply timing of the gate drive signal Vgcl” with the “shift of the supply timing of the selection signal ASW”. The same concept enables the calculation of the pulse wave velocity between different two points that are not illustrated. One of the components employed as the different two points in the detection area AA serves as a first optical sensor, and the other thereof serves as a second optical sensor.

Although the above description with reference to FIGS. 7, 8, 14, 15, and 16 exemplifies the case where the gate drive signal Vgcl is supplied to the gate lines GCL at different timings, the present embodiment is not limited thereto. For example, an area including a plurality of the partial detection areas PAA, such as the group area PAG, may be employed as a pulse wave detection point, such as the point P2 or P5 mentioned above. When the pulse wave detection point is the group area PAG, the supply timings of the gate drive signal Vgcl and the supply timings of the selection signal ASW to the partial detection areas PAA included in the group area PAG are unified. That is, outputs from the partial detection areas PAA included in the group area PAG are treated as one combined output. Thus, the temporal shift between points corresponds to the supply timings of the gate drive signal Vgcl and the supply timings of the selection signal ASW to the respective group areas PAG arranged in different positions. The area including the partial detection areas PAA that is employed as the pulse wave detection point is not limited to the group area PAG, and may be, for example, an area including the partial detection areas PAA arranged in one of the first direction Dx and the second direction Dy. That is, the first optical sensor and the second optical sensor may each be one of the partial detection areas PAA or may include a plurality of the partial detection areas PAA.

For example, the output processor 50 calculates the pulse wave. In this case, for example, an output for a predetermined time stored in the storage 46 is given to the output processor 50 through the signal processor 44, and as a result, the output processor 50 detects the amplitude between a peak and a bottom of the output of each of the photodiodes PD to identify the count timing of the pulsation. The output processor 50 uses the above-described approach to correct the temporal shift of each of the photodiodes PD, and calculates the pulse wave velocity based on the relation of the distance between the photodiodes PD with the count timing of the pulsation based on the output of each of the photodiodes PD. Another component may be used to calculate the pulse wave velocity. For example, the output processor 50 may output data representing the output of each of the photodiodes PD obtained on a predetermined period basis, to an external information processing device or information processing circuit. In this case, the external information processing device or information processing circuit calculates the pulse wave velocity.

In the above description, the blood vessel VB is employed to calculate the pulse wave velocity. The type of the blood vessel VB is not limited to a particular type, such as an artery, a vein, or other.

As described above, the detection device 1 of the embodiment includes the first optical sensor (for example, the photodiode PD(1) at the point P5), the second optical sensor (for example, the photodiode PD(M) at the point P2) disposed at a predetermined distance (for example, the distance In) from the first optical sensor, the light sources (for example, the second light sources 62) that emit light to be detected by the first optical sensor and the second optical sensor facing the living body tissue including the blood vessel (for example, the blood vessel VB), and a processor (for example, the output processor 50) that calculates the pulse wave velocity of the blood vessel based on a time-series variation of the output of the first optical sensor, a time-series variation of the output of the second optical sensor, and the predetermined distance. The time-series variation of the output refers to a time-series variation of the output including the amplitude, such as the peak U1, the bottom D1, the peak U2, the bottom D2, and so on, described with reference to FIG. 16. This configuration allows the pulse wave velocity to be obtained.

As control in the embodiment, control can be employed in which the period in which the first optical sensor (for example, the photodiode PD(1) at the point P5) and the second optical sensor (for example, the photodiode PD(M) at the point P2) are reset (reset period Prst), the period in which the light sources are on (effective exposure period Pex), and the period in which the output from the first optical sensor and the output from the second optical sensor are acquired (reading period Pdet) are independent from one another. This control can reduce the amount of correction of the temporal shift in the calculation of the pulse wave velocity.

As the control in the embodiment, control can also be employed in which the period in which the light sources are on (effective exposure period Pex) overlaps the period in which the first optical sensor (for example, the photodiode PD(1) at the point P5) and the second optical sensor (for example, the photodiode PD(M) at the point P2) are reset (reset period Prst) and the period in which the output from the first optical sensor and the output from the second optical sensor are acquired (reading period Pdet). This control can ensure the longer effective exposure period Pex while shortening one period including the reset period Prst, the reading period Pdet, and the effective exposure period Pex. In this case, first reset timing of resetting the first optical sensor (for example, the photodiode PD(1) at the point P5) differs from second reset timing of resetting the second optical sensor (for example, the photodiode PD(M) at the point P2) (refer to FIG. 14). The processor (for example, the output processor 50) corrects the temporal shift between the period in which the first optical sensor detects the light (for example, the effective exposure period Pex(1)) and the period in which the second optical sensor detects the light (for example, the effective exposure period Pex(M)) based on the temporal shift between the first reset timing and the second reset timing (for example, the time InA(M)), and calculates the pulse wave velocity. As a result, the calculation accuracy of the pulse wave velocity can be further improved.

First acquisition timing of acquiring the output from the first optical sensor (for example, the photodiode PD(1) at the point P5) differs from second acquisition timing of acquiring the output from the second optical sensor (for example, the photodiode PD(M) at the point P2) (refer to FIGS. 14 and 15). The processor (for example, the output processor 50) corrects the temporal shift between the time-series variation of the output of the first optical sensor and the time-series variation of the output of the second optical sensor based on the temporal shift between the first acquisition timing and the second acquisition timing (for example, the time InB(M)), and calculates the pulse wave velocity. As a result, the calculation accuracy of the pulse wave velocity can be further improved.

Each of the first optical sensor and the second optical sensor includes a plurality of optical sensors (for example, the group areas PAG). This configuration can easily increase the output of each of the first optical sensor and the second optical sensor.

The wavelength of the second light L62 is in a range from 500 nm to 950 nm. As a result, the pulsation of the blood vessel VB can be better detected.

The processor (for example, the output processor 50) determines an occurrence of the pulse based on a relation of the degree of amplitude of the output in the time-series variation of the output of the first optical sensor (for example, the photodiode PD(1) at the point P5) and the time-series variation of the output of the second optical sensor (for example, the photodiode PD(M) at the point P2) with the predetermined amplitude reference value (for example, the threshold). As a result, a change in the detection of the optical sensor caused by the pulsation of the blood vessel (for example, the blood vessel VB) can be used for detecting the occurrence of the pulse.

The processor (for example, the output processor 50) identifies an occurrence of a peak (for example, the peak U1) or a bottom (for example, the bottom D1) in one cycle of the amplitude included in the time-series variation of the output of the first optical sensor (for example, the photodiode PD(1) at the point P5) and the time-series variation of the output of the second optical sensor (for example, the photodiode PD(M) at the point P2) as the occurrence of one pulse. As a result, the number of times of occurrence of pulse can be more easily counted.

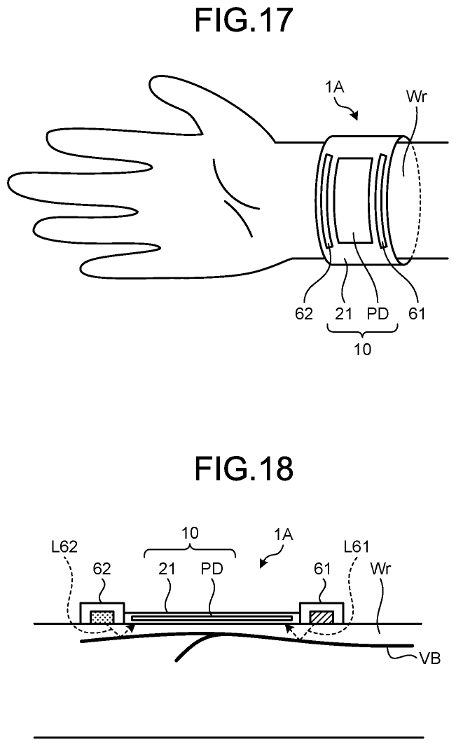

The specific form of the detection device 1 is not limited to the form described with reference to FIGS. 11 and 12. FIG. 17 is a schematic view illustrating a main configuration example of a detection device 1A in a form wearable on a wrist Wr. FIG. 18 is a schematic diagram illustrating an example of the detection of the pulse wave velocity of the blood vessel VB by the detection device 1A illustrated in FIG. 17. As illustrated in FIG. 17, the sensor base member 21 of the detection device 1A has flexibility to be deformable into an annular shape surrounding the wrist Wr. The photodiodes PD, the first light sources 61, and the second light sources 62 are arranged in an arc shape along the annular sensor base member 21.

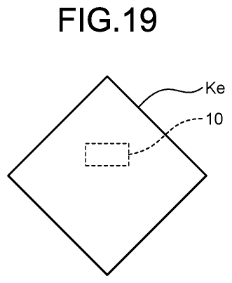

The detection device 1 can be mounted on various products supposed to be in contact with or in proximity to the living body tissue. Mounting examples of the detection device 1 will be described with reference to FIGS. 19, 20, and 21.

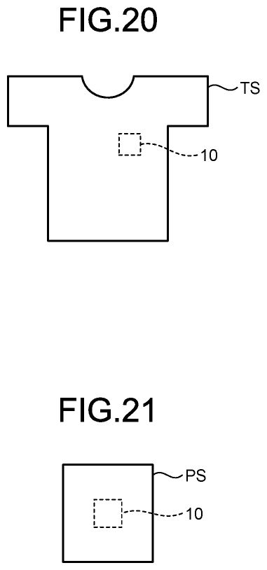

FIG. 19 is a diagram illustrating an arrangement example of the sensor 10 of the detection device 1 mounted on a bandanna Ke. FIG. 20 is a diagram illustrating an arrangement example of the sensor 10 of the detection device 1 mounted on clothes TS. FIG. 21 is a diagram illustrating an arrangement example of the sensor 10 of the detection device 1 mounted on an adhesive sheet PS. For example, the detection device 1 may be incorporated into a product, such as the bandanna Ke of FIG. 19, the clothes TS of FIG. 20, or the adhesive sheet PS of FIG. 21, that is operated to contact the living body tissue. In this case, at least the sensor 10 is preferably provided at a position expected to contact the living body tissue when the product is used. Although not illustrated, the light sources such as the first light sources 61 and the second light sources 62 are preferably arranged taking into account the positional relation between the sensor 10 and the living body tissue. The products are not limited to the bandanna Ke, the clothes TS, and the adhesive sheet PS. The detection device 1 can be incorporated into any product expected to contact the living body tissue when the product is in use. The adhesive sheet PS is a sheet-like product provided with adhesiveness, such as external analgesic and anti-inflammatory sheets.

In the embodiment, the case has been described where the gate line drive circuit 15 performs the time-division selective driving of sequentially supplying the gate drive signals Vgcl to the gate lines GCL. However, the driving method is not limited to this case. The sensor 10 may perform code division selection driving (hereinafter, called “code division multiplexing (CDM) driving”) to perform the detection. Since the CDM driving and a drive circuit thereof are described in Japanese Patent Application No. 2018-005178 (JP-A-2018-005178), what is described in JP-A-2018-005178 is included in the embodiment, and the description will not be omitted herein.

Although the preferred embodiment of the present invention has been described above, the present invention is not limited to the embodiment described above. The content disclosed in the embodiment is merely an example, and can be variously modified within the scope not departing from the gist of the present invention. Any modifications appropriately made within the scope not departing from the gist of the present invention also naturally belong to the technical scope of the present invention.

What is claimed is:

1. A detection device comprising:
a first optical sensor;
a second optical sensor disposed at a predetermined distance from the first optical sensor;
a light source configured to emit light to be detected by the first optical sensor and the second optical sensor facing a living body tissue including a blood vessel; and
a processor configured to calculate a pulse wave velocity of the blood vessel based on a time-series variation of an output of the first optical sensor, a time-series variation of an output of the second optical sensor, and the predetermined distance.

2. The detection device according to claim 1,
wherein a period in which the first optical sensor and the second optical sensor are reset, a period in which the light source is on, and a period in which the output from the first optical sensor and the output from the second optical sensor are acquired are independent from one another.

3. The detection device according to claim 1,
wherein a period in which the light source is on overlaps a period in which the first optical sensor and the second optical sensor are reset and a period in which the output from the first optical sensor and the output from the second optical sensor are acquired.

4. The detection device according to claim 3,
wherein first reset timing of resetting the first optical sensor differs from second reset timing of resetting the second optical sensor, and
wherein the processor is configured to correct a temporal shift between a period in which the first optical sensor detects the light and a period in which the second optical sensor detects the light based on a temporal shift between the first reset timing and the second reset timing, and calculate the pulse wave velocity.

5. The detection device according to claim 2,
wherein first acquisition timing of acquiring the output from the first optical sensor differs from second acquisition timing of acquiring the output from the second optical sensor, and
wherein the processor is configured to correct a temporal shift between the time-series variation of the output of the first optical sensor and the time-series variation of the output of the second optical sensor based on a temporal shift between the first acquisition timing and the second acquisition timing, and calculate the pulse wave velocity.

6. The detection device according to claim 3,
wherein first acquisition timing of acquiring the output from the first optical sensor differs from second acquisition timing of acquiring the output from the second optical sensor, and
wherein the processor is configured to correct a temporal shift between the time-series variation of the output of the first optical sensor and the time-series variation of the output of the second optical sensor based on a temporal shift between the first acquisition timing and the second acquisition timing, and calculate the pulse wave velocity.

7. The detection device according to claim 1,
wherein each of the first optical sensor and the second optical sensor comprises a plurality of optical sensors.

8. The detection device according to claim 1,
wherein a wavelength of the light is in a range from 500 nm to 950 nm.

9. The detection device according to claim 1,
wherein the processor is configured to determine an occurrence of a pulse based on a relation of a degree of amplitude of the output in the time-series variation of the output of the first optical sensor and the time-series variation of the output of the second optical sensor with a predetermined amplitude reference value.

10. The detection device according to claim 9,
wherein the processor is configured to identify an occurrence of a peak or a bottom in one cycle of the amplitude included in the time-series variation of the output of the first optical sensor and the time-series variation of the output of the second optical sensor as an occurrence of one pulse.
